(19) United States
(12) Patent Application Publication
(10) Pub. No.: US 2008/0039931 A1
(43) Pub. Date: Feb. 14, 2008

(54) Hydrophilic shape memory insertable medical articles

(75) Inventors: Bruce M. Jelle, Chanhassen, MN (US); Stephen J. Chudzik, St. Paul, MN (US)

Correspondence Address: KAGAN BINDER, PLLC, SUITE 200, MAPLE ISLAND BUILDING, 221 MAIN STREET NORTH, STILLWATER, MN, 55082, US

(73) Assignee: SurModics, Inc.

(21) Appl. No.: 11/789,786

(22) Filed: Apr. 25, 2007

Related U.S. Application Data
(60) Provisional application No. 60/795,019, filed on Apr. 25, 2006.

(51) Int. Cl. A61F 2/02; A61K 47/42; A61K 8/73; A61K 9/00

(52) U.S. Cl. 623/1.18; 606/78; 424/423; 424/426; 424/486; 424/488; 521/141; 623/1.15; 623/1.22; 623/23.75; 623/4.1

(57) ABSTRACT

Insertable medical articles with a shape memory property having a body member formed of crosslinked hydrophilic polymers are described.

### CROSS-REFERENCE TO RELATED APPLICATION

The present non-provisional patent application claims priority under 35 USC §119(e) from U.S. Provisional Patent Application having Ser. No. 60/795,019, filed on Apr. 25, 2006, and titled HYDROPHILIC SHAPE MEMORY INSERTABLE MEDICAL ARTICLES; and U.S. Provisional Patent Application filed on Apr. 19, 2007, entitled HYDROPHILIC SHAPE MEMORY INSERTABLE MEDICAL ARTICLES, naming inventors Bruce M. Jelle and Stephen J. Chudzik, and having attorney docket number SRM0082/P2; wherein the entirety of said provisional patent applications are incorporated herein by reference.

### FIELD OF THE INVENTION

The present invention relates to insertable medical articles formed from hydrophilic polymers and that have a shape memory property.

### BACKGROUND OF INVENTION

Implantable medical devices, such as stents, have often employed shape memory alloys (SMAs) in their construction. Generally, after a device has been deformed from its original configuration, it regains its original geometry by itself upon heating (one-way effect) or, at higher ambient temperatures, simply during unloading (pseudo-elasticity or superelasticity). These properties are due to a temperature-dependent martensitic phase transformation from a low-symmetry to a highly symmetric crystallographic structure. Those crystal structures are known as martensite (any crystal structure that was formed by displacive transformation, as opposed to much slower diffusive transformations) and austenite.

Shape memory alloys (SMA) form a group of metals that have interesting thermal and mechanical properties. If a SMA material such as NiTinol is deformed while in a martensitic state (low yield strength condition) and then heated to its transition temperature to reach an austenitic state, the SMA material will resume its original (undeformed) shape. The rate of return to the original shape depends upon the amount and rate of thermal energy applied to the component. When the SMA material is cooled, it will return to the martensitic state and shape.

Nickel-titanium (NiTi) are shape memory alloys that are frequently used to fabricate shape memory devices such as vascular prosthesis for the treatment of vascular stenosis. After placement within a body blood vessel and upon heating of the prosthesis to its transition temperature, the prosthesis expands so as to become firmly anchored to the inside wall of the body blood vessel. After expansion the diameter of the lumen of the prosthesis is approximately equal to the diameter of the body blood vessel passageway. The prosthesis may also be used in other body passageways.

While shape memory alloys clearly offer excellent mechanical strength, other properties make them less than ideal for use in the body. One disadvantage is that metals, including nitinol, do not provide an ideal biocompatible surface. Tissue responses to the vascular or coronary placement of metal stents have been studied and generally understood. These tissue responses include phases of attachment of coagulation factors, inflammation and cell recruitment, and proliferation, with the later stages being associated with the presence of endothelial cells (ECs) and smooth muscle cell (SMCs) on the device surface (see, for example, Edelman E. R. and Rogers, C. (1998) Am. J. Cardiol., 81:4 E-6E). However it is commonly seen that the surface of metal stents, the later stages are associated with hyperproliferation of SMCs, leading to hyperplasia and restenosis.

Furthermore, following a desired a period of treatment, metal implantable devices made of alloys such as nitinol are either left in the body, or a surgical procedure is performed to remove the device.

As an alternative to metals, synthetic biodegradable polymers, such as polyglycolide-type molecules, have been used for the construction of implantable medical devices. For example, U.S. Pat. No. 6,991,647 describes self-expanding biodegradable stents that are prepared from a mixture of poly-L-lactide (PLLA) and poly-ε-caprolactone (PCL).

For example, as an alternative to non-biodegradable systems, synthetic biodegradable polymers, such as polyglycolide-type molecules, have been used for the construction of implantable medical devices and for delivery of bioactive agents. These types of polyglycolide-type molecules can degrade into acid products that cause unwanted side effects in the body by virtue of their presence or concentration in vivo. These unwanted side effects can include immune reactions, toxic buildup of the degradation products in the body, or the initiation or provocation of other adverse effects on cells or tissue in the body.

### SUMMARY OF INVENTION

The present invention provides insertable medical articles that have a shape memory property. The insertable medical articles have a body member that includes a matrix of hydrophilic polymers with an internal strength sufficient to revert from a second configuration to a first configuration. The internal strength can be achieved by preparing a body member formed of a matrix comprising crosslinked low molecular weight hydrophilic polymers. Use of low molecular weight hydrophilic polymers allows formation of a dense crosslinked network, providing the body member with a high degree of resiliency. In addition to this resiliency, the articles are remarkably compliant, and are therefore resistant to detrimental fracturing or cracking that may otherwise occur as a result of manipulating the body member of the article from one configuration to another. The internal strength allows the body member to revert to a first configuration following release from a second configuration.

In some aspects, the invention provides an insertable medical article comprising a body member comprising a crosslinked matrix of hydrophilic polymer having a molecular weight of 100,000 Da or less, wherein the body member is capable of undergoing a shape memory transition from a second configuration to a first configuration upon insertion of the article at a target site in a subject.

The invention also provides a method for preparing an insertable medical article comprising a body member having a shape memory property. The method of preparing the article comprises steps of (a) providing a composition in a first configuration, the composition comprising a hydrophilic polymer having a molecular weight of 100,000 Da or less, and a reactive group, and (b) causing reaction of the reactive group thereby forming a matrix of hydrophilic polymer and fabricating the body member in the first configuration. The method forms a body member with an internal strength sufficient to revert to the first configuration from a second configuration.

In some cases, the hydrophilic polymer comprises a reactive group that is a pendent polymerizable group, and the polymerizable group is activated to cause polymerization of the hydrophilic polymer, thereby forming the matrix.

In some cases, the hydrophilic polymer comprises a reactive group that is a first reactive group pendent from the hydrophilic polymer. The matrix further comprises a second component that is hydrophilic and that comprises a second reactive group. The first and second groups are specifically reactive and provide a crosslinked hydrophilic polymeric matrix upon mixing, thereby fabricating the body member in the first configuration.

In some aspects, the invention provides an insertable medical article comprising a body member formed of a hydrophilic matrix comprising a first polymeric network that penetrates a second polymeric network (an “inter penetrating network”). The article can be formed from a first component that is a hydrophilic polymer comprising a first reactive group, a second component that is hydrophilic and that comprises a second reactive group, and a third component that is a hydrophilic polymer comprising a pendent polymerizable group. The first and second groups are specifically reactive, and are combined to generate a first polymeric network. The third component is polymerized to form a second polymeric network that penetrates the first polymeric network. The first and second polymeric networks are formed to provide the body member in the first configuration, and the body member is capable of reverting to the first configuration from a second configuration.

In some aspects, the body member can be in a second configuration that is linear. In reverting to the first configuration, the body member can assume a non-linear configuration. The non-linear configuration can comprise a curve, such as a coil configuration, which can be therapeutically useful to a subject at a target site. The target site can be any selected site in the body, such as an intravascular location, or a portion of the eye.

The body member can be in any desired form, and the form can be in two or more configurations, including the first and second configuration. For example, the body member can be in the form of a filament, a sheet, or a cylinder. For example, a first configuration of a filament can be coiled and a second configuration of the filament can be linear. A cylinder can be in a first configuration that is expanded and a second configuration that is collapsed.

In some aspects, the second configuration is maintained by physically constraining the body member in the second configuration. For example, the body member can be constrained by confining it within an insertion instrument. Upon release of the article from the insertion instrument at a target location, the body member can revert to the first configuration.

In this aspect, the invention provides an insertable medical article having a body member, wherein the body member comprises a matrix of hydrophilic polymer, and the body member has an internal strength and is capable of undergoing a shape memory transition from a second configuration to a first configuration.

The body member in the second configuration can facilitate its delivery to a target location within the body. For example, the body member can be delivered through the vasculature, or through a portion of the eye to a target site. In some aspects, the second configuration of the body member can be of a dimension or shape that allows it to be retained within a insertion instrument, which is used to deliver the shape memory article to a target site where it is released and reverts to the first configuration. The second configuration of the body member can be of a dimension or shape that allows it to be passed though a portion of the body that would otherwise not allow passage of the article in the first configuration.

In some aspects, the invention provides a method for treating a target site within a subject where the body member in the first configuration exerts force on a target tissue. The method can include the steps of (a) obtaining an insertable medical article comprising a body member comprising a matrix of hydrophilic polymer, wherein the body member is capable of undergoing a shape memory transition from a second configuration to a first configuration; and (b) delivering the article to a target site within a subject in the second configuration; allowing the body member to revert from the second configuration to the first configuration at the target site, wherein the body member in the first configuration exerts force on a tissue at the target site.

In other aspects, the body member is maintained in a second configuration in a dehydrated state. The dehydrated state may allow the body member to be held in the second configuration without physically constraining the body member (in the second configuration). Upon rehydration, the body member reverts to the first configuration. Since rehydration drives reconfiguration of the body member, such an insertable article is herein referred to as having a “hydration-based shape memory” property. In this aspect, the invention provides an insertable medical article comprising a body member comprising a matrix of hydrophilic polymeric material in a second configuration. The body member in the second configuration has a second hydration state and is capable of undergoing a shape memory transition to a first configuration. Transition to a first configuration can occur upon increasing the hydration of the body member to a first hydration state.

In some aspects, the invention provides an insertable medical article comprising a body member comprising a crosslinked matrix of hydrophilic polymer having a molecular weight of 100,000 Da or less, wherein the body member is in a second configuration having a second hydration state, and is capable of undergoing a shape memory transition to a first configuration upon insertion of the article at a target site in a subject which increases the hydration of the body member to a first hydration state.

In some aspects, the invention provides a method for preparing an insertable medical article comprising a body member having a hydration state-based shape memory property. In this aspect, the method includes the steps of (a) providing a composition comprising a hydrophilic polymer having a molecular weight of 100,000 Da or less, a reactive group, and liquid; (b) causing reaction of the reactive group thereby forming a matrix of hydrophilic polymer and fabricating the body member in the first configuration; (c) reconfiguring the body member from the first configuration to a second configuration; and (d) removing at least a portion of liquid to stabilize the body member in the second configuration.

The step of removing can involve removing about 50% or greater of the liquid (e.g., water) from the body member. The body member is temporarily fixed in this second configuration (e.g., in a dehydrated from) and will not revert to the first configuration until it is rehydrated.

In some aspects, the invention provides another method for treating a target site within a subject where the body member in the first configuration exerts force on a target tissue. The method can include the steps of (a) obtaining an insertable medical article comprising a body member having a hydration state-based shape memory property, wherein the body member comprises a matrix of hydrophilic polymer; (b) delivering the article to a target site within a subject in a second configuration; and (c) allowing the body member of the article to become hydrated to promote change to a first configuration.

The novel articles of the invention are advantageous for use within the body as shape memory prosthetic devices. Many prosthetic devices, such as stents, are designed to reside within a vessel lumen and exert force against tissue of the lumen wall. Given that the body member of the shape memory article is constructed from hydrophilic polymeric material, it can have improved biocompatibility as compared to other prosthetic devices that are constructed from metal or other non-hydrophilic polymeric materials. The improved biocompatibility can in turn lead to a decrease in adverse tissue responses in the target area and a decrease in the occurrence of restenosis. This can improve the functional life of the prosthetic device.

The hydrophilic polymers used to form the body member can be biostable or biodegradable. In some aspects, the body member of the insertable article is fabricated from a biodegradable hydrophilic polymeric material. Biodegradable implantable medical articles such as stents can be fabricated to have a desired in vivo functional life. After placement at a target site, an implantable article with a predetermined in vivo functional life can perform a function for a period of time and then degrade. This eliminates the need for the article to be removed from the body after the desired period of use. For example, in some aspects, the implantable articles are prepared to have an in vivo functional life of about up to about 6 months, or in the range of about 2 to about 6 months.

In some aspects of the invention, the article includes a biodegradable body member that comprises matrix of natural biodegradable polysaccharides. In addition to the resilient and compliant properties of the body member, the biodegradable body member can also have improved degradation qualities. Biodegradable shape memory articles prepared using biodegradable polysaccharides can have the advantage of degrading by surface erosion, as opposed to bulk erosion which is common to other biodegradable polymers.

This can be beneficial in many regards. For example, in some aspects, as a natural biodegradable polysaccharide-based shape memory article degrades, it gradually looses its mechanical strength. This results in a slower transfer of stress to the surrounding tissues, which may be of greater benefit for treating a particular medical condition. Furthermore, since these articles degrade by surface erosion, this removes the risk that degraded particulates present a risk of migrating to a secondary location and acting as emboli. In these aspects, biodegradable polysaccharides also offer the advantage of breaking down into inert degradation products, such as glucose. These naturally occurring mono- or disaccharides are common serum components and present little or no immunogenic or toxic risk to the individual.

The shape memory article can also include a bioactive agent. In the case of a biodegradable matrix, the bioactive agent can be released upon degradation of one or more portions of the body member. In this regard, the body member optionally may exert a force on a target tissue, but it is not required.

In some aspects the bioactive agent is selected from the group consisting of polypeptides, polynucleotides, and polysaccharides. In some aspects, the insertable medical article comprises a bioactive agent having a molecular weight of 10,000 or greater.

In other aspects, the body member includes microparticles, and the microparticles include a low molecular weight bioactive agent. The use of microparticles can represent one method of controlling the release of a low molecular weight bioactive agent from the body member of the shape memory article.

In some aspects, the insertable medical article includes an anti-thrombotic agent. For example, the insertable medical article can include heparin. Heparin can be used in a shape memory article such as a stent to prevent thrombus formation in the vicinity of the stent.

In some aspects, the insertable medical article includes a pro-thrombotic agent. For example, the insertable medical article can include collagen. Collagen can be used in a shape memory article such as an occlusion device to promote a thrombogenic response in the vicinity of the shape memory article.

The method also provides methods for delivering a bioactive agent to a subject. The method can include the steps of (a) obtaining an insertable medical article comprising a body member comprising a matrix of hydrophilic polymer and a bioactive agent, wherein the body member is capable of undergoing a shape memory transition from a second configuration to a first configuration; (b) delivering the article to a target site within a subject in the second configuration; (c) allowing the body member to revert from the second configuration to the first configuration at the target site; and (d) allowing release of the bioactive agent from the body member at the target site.

In some aspects the method provides methods for delivering a bioactive agent to a subject using the article in dehydrated form. The method can include the steps of (a) obtaining an insertable medical article comprising a body member having a hydration state-based shape memory property, wherein the body member comprises a matrix of hydrophilic polymer and a bioactive agent; (b) delivering the article to a target site in the body in a second configuration; (c) allowing the body member to become hydrated to promote reversion to a first configuration; and (d) allowing release of the bioactive agent from the body member at the target site.

The invention also provides systems for delivering to a portion of the body an insertable medical article comprising a body member having a shape memory property. The system comprises an insertable medical article comprising a body member comprising a crosslinked matrix of hydrophilic polymer, wherein the body member is capable of undergoing a shape memory transition from a second configuration to a first configuration upon insertion of the article at a target site in a subject. The system also comprises an insertion instrument capable of holding the article in a second configuration. The system can be provided where the shape memory article is in a second configuration and loaded into a portion of the insertion instrument.

### BRIEF DESCRIPTION OF THE DRAWINGS

FIG. 1 is an illustration of a mold for forming a shape memory coil, the mold having flexible tubing wrapped around a mandrel.

FIG. 2 is an illustration of a shape memory coil in a first configuration.

FIG. 3 is an illustration of a circular cross section of a shape memory coil.

FIG. 4a is an illustration of a shape memory coil reconfigured into a second (linear) configuration.

FIG. 4b is an illustration of a shape memory coil reconfigured into a second (tightly coiled) configuration.

FIG. 5 is an illustration of a shape memory cylinder (stent) with fenestrations in a first (expanded) configuration.

FIG. 6a is an illustration of a cross section of a shape memory cylinder (stent) in a first (expanded) configuration.

FIG. 6b is an illustration of a cross section of a shape memory cylinder (stent) in a second (collapsed) configuration.

FIG. 6c is an illustration of a cross section of a shape memory cylinder (stent) in a second (collapsed) configuration.

FIG. 6d is an illustration of a cross section of a shape memory cylinder (stent) in a second (collapsed) configuration.

FIG. 7a is an illustration of a shape memory cylinder (stent) with a slit in a first (expanded) configuration.

FIG. 7b is an illustration of a shape memory cylinder (stent) with a slit in a second (rolled) configuration.

FIG. 8 is a reaction scheme showing the preparation of an amine-functional polysaccharide.

FIG. 9 is a reaction scheme showing the preparation of an amine-reactive compound.

FIG. 10 is reaction scheme showing the preparation of an amine-reactive compound.

FIG. 11 is a reaction scheme showing the formation of a matrix material by the reaction of an amine-functional polysaccharide with an amine reactive compound.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention provides insertable medical articles that have a shape memory property, which have body members formed of low molecular weight hydrophilic polymers. The body member is formed in a first configuration, and is capable of reverting to the first configuration following release from a second configuration (which is different than the first configuration).

A change in “configuration” refers to a fundamental change in the shape of the article, as exemplified by a change from a linear configuration to a non-linear configuration, or vise versa. This change is not merely an expansion of a particular configuration, as may be seen when a hydrogel swells upon contact with water and expands in size in a first configuration. Rather, the insertable medical article may be described as having first and second ends, and in the first configuration the path (such as a linear or non-linear path) between the first and second ends defines a shape of the article. In the second configuration, the path between the first and second ends is different than in the first configuration.

In some cases, when the insertable medical article changes from a second configuration to a first configuration, the body member may optionally expand in size. This expansion, accompanied by a change in configuration, may occur if the body member in the second configuration is in a dehydrated state and the hydration state increases in the first configuration.

According to the invention, the body member of the shape memory article is prepared from a hydrophilic polymer that can have a low molecular weight. Use of low molecular weight polymers allow for the preparation of a body member having a matrix with a high density of polymer and good internal strength. Generally, the molecular weight of the hydrophilic polymer is 100,000 Da or less. Preferably the molecular weight is 50,000 Da or less, 25,000 Da or less, or 10,000 Da or less. A particularly preferred size range for the hydrophilic polymer is in the range of about 1000 Da to about 10,000 Da. Another preferred size range for the hydrophilic polymer is in the range of about 1000 Da to about 5,000 Da.

In some preparations the hydrophilic polymer is degradable. These can be used to form a shape memory article that is entirely or partially degradable. Some preferred degradable hydrophilic polymers are natural biodegradable polysaccharides. As referred to herein, a “natural biodegradable polysaccharide” refers to a non-synthetic polysaccharide that is capable of being enzymatically degraded but that is generally non-enzymatically hydrolytically stable. Natural biodegradable polysaccharides include polysaccharide and/or polysaccharide derivatives that are obtained from natural sources, such as plants or animals. Natural biodegradable polysaccharides include any polysaccharide that has been processed or modified from a natural biodegradable polysaccharide (for example, maltodextrin is a natural biodegradable polysaccharide that is processed from starch).

If a natural biodegradable polysaccharide is used to form the body member of the shape memory article, it is desirably a low molecular weight polymer. Exemplary natural biodegradable polysaccharides include low molecular weight preparations of amylose, maltodextrin, cyclodextrin, polyalditol, hyaluronic acid, starch, dextran, heparin, chondroitin sulfate, dermatan sulfate, heparan sulfate, keratan sulfate, dextran sulfate, pentosan polysulfate, and chitosan. The natural biodegradable polysaccharide can be a substantially non-branched or completely non-branched poly(glucopyranose) polymer.

As used herein, “amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4%-5% of the total linkages. Generally amylopectin derived from plant sources have molecular weights of 1×10⁷ Da or greater.

Amylose can be obtained from, or is present in, a variety of sources. Typically, amylose is obtained from non-animal sources, such as plant sources. In some aspects, a purified preparation of amylose is used as starting material for the preparation of the amylose polymer having coupling groups, which can be used to form the body member of the shape memory article. In other aspects, as starting material, amylose can be used in a mixture that includes other polysaccharides.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate×100.

A starch preparation that has been totally hydrolyzed to dextrose (glucose) has a DE of 100, where as starch has a DE of about zero. A DE of greater than 0 but less than 100 characterizes the mean-average molecular weight of a starch hydrolysate, and maltodextrins are considered to have a DE of less than 20. Maltodextrins of various molecular weights, for example, in the range of about 500-5000 Da are commercially available (for example, from CarboMer, San Diego, Calif.).

Another contemplated class of natural biodegradable polysaccharides is natural biodegradable non-reducing polysaccharides. An exemplary non-reducing polysaccharide comprises polyalditol, which is available from GPC (Muscatine, Iowa), which has a non-reducing terminus (lacking an aldehyde group). A non-reducing polysaccharide can provide an inert matrix thereby improving the stability of sensitive bioactive agents (if included in the body member of the shape memory article), such as proteins and enzymes. A non-reducing polysaccharide can include a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). In another aspect, the polysaccharide is a glucopyranosyl polymer, such as a polymer that includes repeating (1→3)O-β-D-glucopyranosyl units.

In some preferred aspects of the invention, the body member of the shape-memory article comprises biodegradable polysaccharide selected from maltodextrin, amylose, polyalditol, and cyclodextrin. For example, the body member can be formed by a composition that includes a biodegradable polysaccharide selected from maltodextrin, amylose, polyalditol, and cyclodextrin, wherein the polysaccharide comprises pendent polymerizable groups, and wherein the polysaccharide has a molecular weight of 10,000 Da or less. As another example, the body member can be formed by a composition that includes a biodegradable polysaccharide selected from maltodextrin, amylose, polyalditol, and cyclodextrin, wherein the polysaccharide comprises pendent first reactive groups of a reactive pair, and wherein the polysaccharide has a molecular weight of 10,000 Da or less.

In some aspects, the body member of the shape memory article includes a hydrophilic synthetic degradable polymer. The synthetic degradable polymer may be fully biodegradable or partially degradable. For example, a partially biodegradable synthetic polymer can include biodegradable segments and non-biodegradable segments.

Some examples of degradable hydrophilic synthetic polymers that include hydrophilic monomers are described in U.S. Pat. No. 5,410,016. These polymers include hydrophilic poly(ethylene glycol) oligomers with biodegradable poly(α-hydroxy acid) extensions and acrylate-type monomer or oligomer end caps.

Dextran-based copolymers (e.g., methacrylate-modified dextran) can also be in the preparation of the body member. Examples of dextran-based polymers are described in U.S. Pat. Nos. 6,303,148 and 6,805,879.

Other degradable hydrophilic synthetic polymers include homopolymers of polyanhydrides, as prepared from aromatic diacids that were acetylated and modified with ethylene glycol segments (Biomaterials (2005) 26:721-8).

In some aspects of the invention, the body member comprises a non-biodegradable hydrophilic polymer. Exemplary non-biodegradable polymers include poly(ethylene oxide) (PEO), polyvinyl alcohol (PVA), poly(vinyl pyrrolidone) (PVP), poly(ethylene glycol), polyacrylamide, poly(hydroxy alkyl methacrylates), poly(hydroxy ethyl methacrylate), hydrophilic polyurethanes, HYPAN, oriented HYPAN, poly(hydroxy ethyl acrylate), poly(ethyloxazoline), and poly(propylene oxide) (PPO).

In some aspects the body member is formed from a composition that includes a non-biodegradable hydrophilic polymer, wherein the hydrophilic polymer comprises pendent polymerizable groups, and wherein the hydrophilic polymer has a molecular weight of 10,000 Da or less.

Additional hydrophilic components can be used to form the body member of the shape memory article. For example, in addition to a hydrophilic polymer having a pendent reactive group, a second component that is a hydrophilic can be used to form the matrix of the body member. The second component can include a second reactive group that can be a polymerizable group, or a member of a reactive pair (such as one that would react with a first reactive group pendent from the hydrophilic polymer). The second component can be a hydrophilic polymer. If used, the second component that is a hydrophilic polymer can be degradable or non-degradable. Other additional hydrophilic components can be used to form the body member, such as lower molecular weight non-polymeric hydrophilic components.

Therefore, the body member can also be formed from blends of two or more hydrophilic polymers that include pendent coupling groups that can be crosslinked to form body member of the shape memory article of the invention. The blends may include mixtures of polymers of different molecular weights, or mixtures of different types of polymers.

Compositions including a hydrophilic polymer (such as a natural biodegradable polysaccharide) and that are used to form the body member can be prepared at particularly high concentrations, such as about 250 mg/mL or greater, or 300 mg/mL or greater. Body members prepared from these compositions can therefore have a high degree of internal strength.

A body member having a matrix of hydrophilic polymer can be formed by using composition including a hydrophilic polymer with pendent coupling groups. These coupling groups can be activated when the composition is in a desired first configuration. Activation of the coupling groups promotes polymer crosslinking and allows formation of the body member in the first configuration. In the first configuration the body member has an internal strength based on the formed matrix and is able to revert to the first configuration upon release from a second configuration.

Various synthetic procedures can be used to provide coupling groups pendent from the hydrophilic polymer (e.g., groups that are reactive and allow crosslinks between the hydrophilic polymers). These procedures can provide a desired number of coupling groups pendent from the hydrophilic polymer, such as a natural biodegradable polysaccharide backbone. For example, hydroxyl groups on the polymer can be reacted with a coupling group-containing compound or can be modified to be reactive with a coupling group-containing compound. The number and/or density of coupling groups, such as acrylate groups, can be controlled using the present method, for example, by controlling the relative concentration of reactive group to hydroxyl group content

In preferred aspects the matrix is formed using coupling groups that allow hydrophilic polymers to be crosslinked, wherein the crosslinks include covalent bonds. A “coupling group” can include (1) a chemical group that is able to form a reactive species that can react with the same or similar chemical group to form a bond that is able to couple the hydrophilic polymers together (for example, wherein the formation of a reactive species can be promoted by an initiator); or (2) a pair of two different chemical groups that are able to specifically react to form a bond that is able to couple the hydrophilic polymers together. The coupling group can be attached to any suitable hydrophilic polymer, including biodegradable and non-biodegradable polymers.

Contemplated reactive pairs include Reactive Group A and corresponding Reactive Group B as shown in the Table 1 below. For the preparation of a composition for formation of the body member, a reactive group from group A can be selected and pendent from a first set of hydrophilic polymers and a corresponding reactive group B can be selected and pendent from a second set of hydrophilic components, such as hydrophilic polymers. Reactive groups A and B can represent first and second coupling groups, respectively. At least one and preferably two, or more than two reactive groups are pendent from an individual hydrophilic polymer or hydrophilic component. The first and second sets of hydrophilic polymers or components can be combined and reacted, for example, thermochemically, if necessary, to promote the coupling of hydrophilic polymers/components and the formation of the body member of the shape memory article.

TABLE 1

| Reactive group A | Reactive group B |
|---|---|
| amine, hydroxyl, sulfhydryl | N-oxysuccinimide (“NOS”) |
| amine | Aldehyde |
| amine | Isothiocyanate |
| amine, sulfhydryl | Bromoacetyl |
| amine, sulfhydryl | Chloroacetyl |
| amine, sulfhydryl | Iodoacetyl |
| amine, hydroxyl | Anhydride |
| amine, hydroxyl | Imidazole carbamate |
| aldehyde | Hydrazide |
| amine, hydroxyl, carboxylic acid | Isocyanate |
| amine, sulfhydryl | Maleimide |
| sulfhydryl | Vinylsulfone |

Amine also includes hydrazide (R—NH—NH₂)

For example, a suitable coupling pair would be a hydrophilic polymer having an electrophilic group and a hydrophilic polymer having a nucleophilic group. An example of a suitable electrophilic-nucleophilic pair is N-hydroxysuccinimide-amine pair, respectively. Another suitable pair would be an oxirane-amine pair.

Accordingly, in some modes of preparation, the hydrophilic matrix can be formed by at least a hydrophilic polymer having two or more first reactive groups (described herein as the “A component”), and a hydrophilic component that includes two or more second reactive groups (described herein as the “B component”). Upon mixing, the first and second groups specifically react, coupling the hydrophilic polymer to the hydrophilic component and forming a crosslinked hydrophilic matrix.

Various A-B combinations can be used. For example, the A component can either be a degradable or a non-degradable hydrophilic polymer. In some cases, the B component is the same as the A component, with the exception that the second reactive group is different than the first reactive group. For example, both the A and B components can be natural degradable polysaccharides. The matrix can be formed predominantly or entirely of the A and B degradable components.

In some preferred modes of preparing the body member, the matrix is formed from an A component that is degradable and a B component that is non-degradable. For example, the B component can be a non-degradable hydrophilic polymer, or a non-degradable hydrophilic non-polymeric compound. Use of these components results in a crosslinked matrix of biodegradable and non-biodegradable components. The shape memory article is degraded in vivo by degradation of the A components, such as by enzymatic degradation. When a natural biodegradable polysaccharide is used as the A component, the implanted article can be degraded in vivo by surface erosion resulting in loss of the matrix components from the surface of the article. Upon sufficient degradation of the A components, the non-degradable B components are lost from the matrix and can be excreted from the body.

In one preparation, the A component is a hydrophilic polymer that is a natural degradable polysaccharide, such as maltodextrin, polyalditol, or amylose. To prepare the A component, and as an example, a portion of the hydroxyl groups of the natural degradable polysaccharide are derivatized with first reactive groups that are amine groups to provide an aminated polysaccharide. Various reaction schemes known in the art can be used to provide a natural degradable polysaccharide with pendent amine groups. In one mode of practice, the polysaccharide is subjected to a two-step reaction scheme to provide pendent amine-reactive groups. In a first step the polysaccharide is reacted with a hydroxyl reactive compound to provide a linking group, to which an amine-containing compound is reacted, providing amine groups that are pendent from the polysaccharide and which represent the first reactive group. In the first step, the hydroxyl-reactive compound is used at a concentration to provide a desired degree of substitution on the polysaccharide.

In some aspects, a non-reducing polysaccharide is used to form the A component. For example, the non-reducing polysaccharide is polyalditol, which has a non-reducing terminus (e.g, the polysaccharide does not have an aldehyde group on its terminal end).

Non-reducing polysaccharides are preferred when amine groups are introduced or present on either the A or B component, as they do not contain pendant aldehyde groups. Pendant aldehyde groups may react with the pendant amine groups on the amine-functional polysaccharide and cause a reduction in the reactivity and/or shelf-life of the amine-functional polysaccharide.

An example of a hydroxyl reactive compound that provides a linking group for this type of synthesis is 1,1′-carbonyldiimidazole (CDI). CDI is useful linking group because it reacts to form a carbamate ester with a hydroxyl group that is present on the natural biodegradable polysaccharide. Once CDI reacts with a first hydroxyl group on the natural biodegradable polysaccharide to form a carbamate ester, the reactivity of the pendant imidazole carbamate group to a second hydroxyl groups is significantly reduced. This is advantageous, because the pendant imidazole carbamate group can remain as an unreacted pendant group from the polysaccharide, and can be used to form a covalent bond to another molecule, typically a more reactive active-hydrogen compound such as an amine.

Following reaction, the polysaccharide is provided with pendent reactive imidazole carbamate groups that can be further reacted with a compound to provide pendent amine groups. In many embodiments of the invention, the pendant imidazole carbamate group is reacted with an amine-containing compound in order to form a polysaccharide having pendent amine groups. For example, the imidazolyl carbamate groups are reacted with a compound having at least two amine groups (e.g., a diamine). In the case of a diamine, one of the amine groups reacts with the imidazole carbamate linking group and, following reaction, the other amine group becomes pendent from the polysaccharide and represents the first reactive group.

Amine-containing compounds that have two or more primary amine groups that are separated by a linking group, such as an alkyl group, can be reacted with the polysaccharide intermediate. In some embodiments, the amine-containing compound has the general formula H₂N—R—NH₂, where R is a straight or branched chain alkyl group. Representative examples of multifunctional amine compounds include 1,6-diaminohexane, 1,4-diaminobutane, 1,3-diaminopropane, and the like.

Generally, the imidazole-functional polysaccharide is reacted with an excess of the diamine compound to ensure there is little or no reaction of a single diamine with two imidazole linking groups. For example, in many embodiments, the imidazole-functional polysaccharide is slowly added to a solution containing the amine-containing compound in order to provide reaction conditions where the amine-containing compound is in substantial excess relative to the imidizole-functional polysaccharide.

The reaction scheme described above may be varied in order to produce aminated polysaccharides having varying degrees of substitution (DS). As used herein the term “degree of substitution” generally refers to the number of hydroxyl groups, on average, per glycopyranose monomeric residue that are derivitized (a polysaccharide such as maltodextrin has three hydroxyl groups per monomeric residue; maltodextrin having a DS1 has approximately 1 hydroxyl group per monomeric residue substituted). In some embodiments, the degree of substitution (DS) of the polysaccharide ranges from about 0.1 to about 1.0. In more preferred embodiments, the degree of substitution ranges from about 0.2 to about 0.3, although other degrees of substitution may be desirable. In an exemplary embodiment, polyalditol is reacted with CDI followed by 1,6-diaminohexane in order to produce an aminated polyalditol having a degree of substitution ranging from about 0.2 to about 0.3.

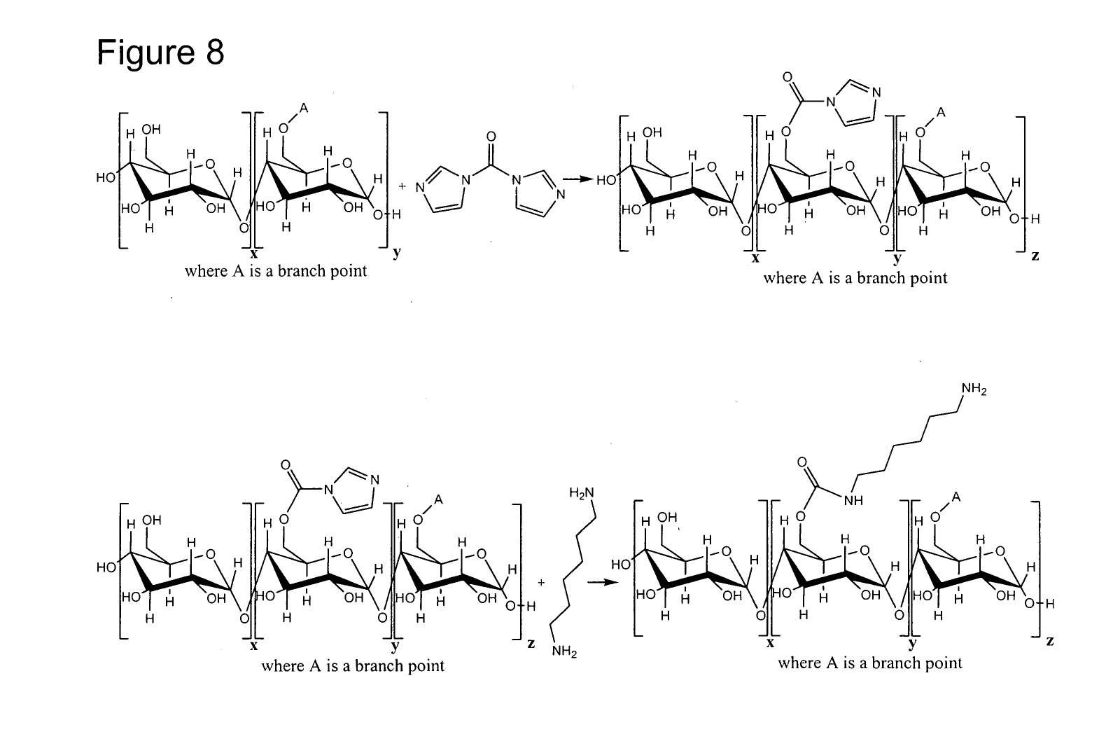

The reaction of polyalditol with CDI, followed by reaction with the amine-containing compound 1,6-diaminohexane is shown in FIG. 8.

After reacting with an excess of amine-containing compound, the resulting aminated polysaccharide is typically purified in order to remove any unreacted amine. Purification techniques include, for example, recrystallization (e.g., using THF), and other precipitation methods and/or dialysis.

In some aspects of the invention, the body member of the shape memory article is formed by the reaction of at least component A that comprises an aminated natural biodegradable polysaccharide, and component B that comprises an amine-reactive hydrophilic compound.

As an example, and for use with an aminated polysaccharide, Component B can be any suitable hydrophilic biocompatible compound that has two or more amine-reactive groups (i.e., the second reactive groups).

In one mode of preparation, component B is a hydroxyl-containing compound that is chemically modified in order to introduce amine-reactive functional groups. Desirably, hydroxyl-containing compounds having at least two pendant hydroxy groups (typically 2 to 4), having biocompatibility, having appreciable water-solubility, and having a molecular weight of about 10,000 Da or less are used for the synthesis of the B component.

In many embodiments, the hydroxyl groups are present as pendant groups from a hydrophilic compound having a hydrophilic organic backbone that comprises atoms of carbon, hydrogen, and oxygen. In some embodiments, the organic backbone is an alkoxyalkane backbone. Representative examples of hydrophilic compounds of this type include polyalkoxyalkane such as poly(ethylene glycol), tetraethylene glycol, triethylene glycol, trimethylolpropane ethoxylate, and pentaeerythritol etholxylate. In some aspects the hydrophilic compound is a liquid at about room temperature (˜25° C.). In many embodiments, a preferred hydroxyl-containing compound is an ethylene glycol polymer or oligomer having the structure HO—(CH₂—CH₂—O)ₙ—H. Typically, the value of n ranges from about 3 to about 150 and the number average molecular weight (Mn) of the poly(ethylene glycol) ranges from about 100 Da to about 5000 Da, more typically ranging from about 200 Da to about 3500 Da.

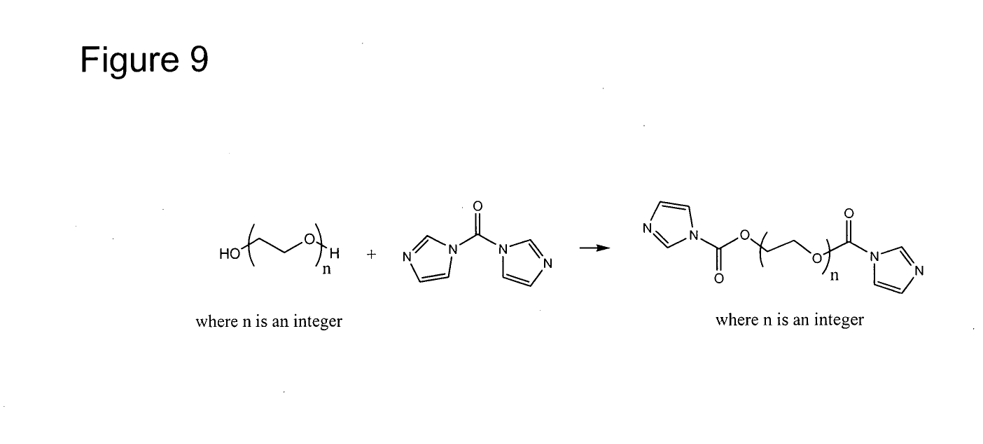

Various synthetic schemes can be used to provide the hydrophilic compound with amine reactive groups. In one mode of practice, the amine-reactive compound is formed by reacting the hydroxy functional compound with 1,1′-carbonyldiimidazole (CDI). The compound 1,1′-carbonyldiimidazole reacts with the hydroxyl groups on the hydrophilic compound resulting in the formation of pendant imidazole carbamate groups, which represent second reactive groups. The reaction of a poly(ethylene glycol) with CDI to produce an amine-reactive compound is shown in FIG. 9.

The pendant imidazole carbamate groups are reactive with amine groups, such as the amine groups that are present on the aminated polysaccharide described hereinabove.

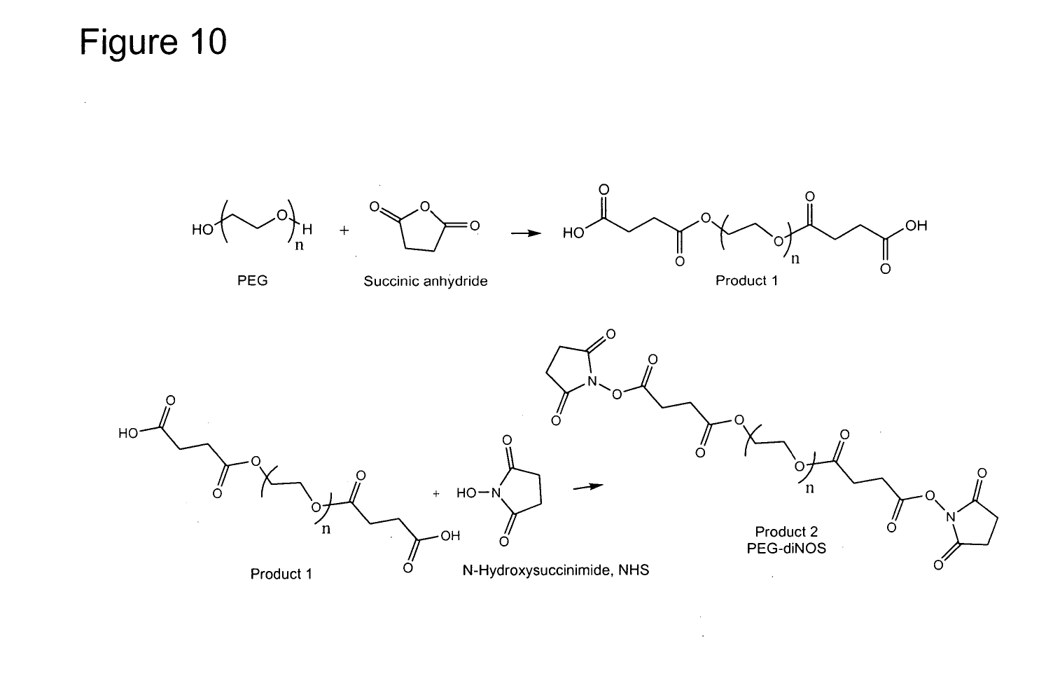

As another example of preparation of a B component having second reactive groups, an amine-reactive compound is prepared by first reacting succinic anhydride with a polyol (e.g., a diol, triol, or tetrol) to form a multi-functional carboxylic acid compound. The succinic anhydride reacts with the hydroxyl groups on the polyol to form an ester linkage and a terminal carboxylic acid group. The multifunctional carboxylic acid compound is then reacted with N-hydroxysuccimide (NHS) which reacts with the terminal carboxylic acid groups to form an amine-reactive NOS groups. In an exemplary embodiment, polyethylene glycol is reacted with succinic anhydride to form a dicarboxylic acid compound (see, FIG. 10, Product 1). The dicarboxylic acid compound is then reacted with N-hydroxysuccimide (NHS) in order to form an amine-reactive compound having two terminal NOS groups (see, FIG. 10, Product 2).

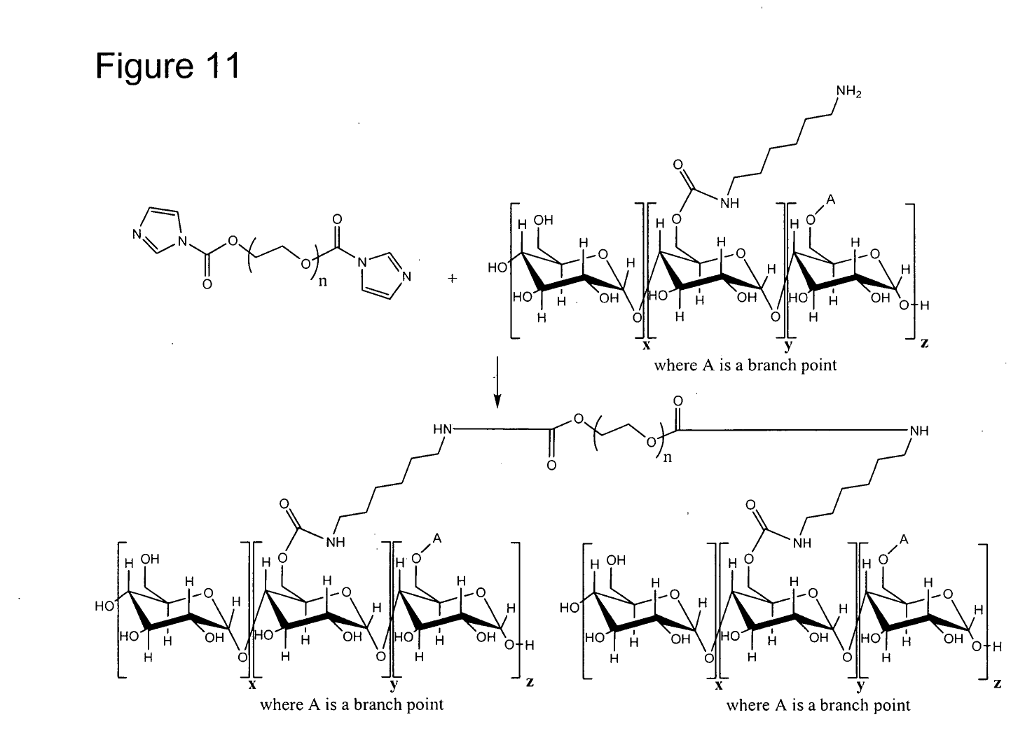

In view of the reactive nature of the first component and the second component, these components are typically held in separate containers from one another until prior to the time that formation of the matrix making the body member of the shape memory article is desired. When the formation of the matrix is desired, the A and B components are mixed with one another in the desired ratio to initiate formation of the matrix, as exemplified by reaction of an amine-containing polysaccharide with an amine-reactive alkoxyalkane. Reaction of the first and second components with one another results in the formation of the enzymatically degradable matrix, which forms the body member of the shape memory article. For example, the reaction of the product of FIG. 8 with the product of FIG. 9 is shown below in FIG. 11.

Typically, the A and B components are reacted with one another in a desired stoichiometric ratio in order to form the matrix. As one way of describing the amount of components A and B used to form the matrix, a particular stoichiometric ratio of the number of moles of amine groups in the amine-functional polysaccharide to the number of moles of amine-reactive groups in the amine-functional compound is used. For example, the stoichiometric ratio of amine groups to amine-reactive groups can be in the range from about 1:5 to about 5:1.

After initiating the formation of the matrix material by reacting the A component with the B component, the components typically cure to form the matrix in a time period that ranges from about a minutes to several hours. More typically, the components cure to form the matrix material in a time period that ranges from about 1 to about 60 (minutes).

The cure time of a given formulation of the matrix material may be adjusted to prepare a particular body member, or suit the particular process that is used to form the body member. For example, the body member may be formed in a particular mold or by a particular process that would be facilitated by using a composition (the mixture of the A and B components) that has a relatively slow cure rate. One method of adjusting the rate of reaction is to control the pH of the composition that includes the mixture of the A and B components. Generally speaking, for chemistries using first and second groups that are amine and amine-reactive groups, a higher pH will favor a faster reaction rate, whereas a lower pH will favor a slower reaction rate between the first and second components. In most embodiments, the pH is controlled between a lower pH limit of about 7.5 and an upper pH limit of about 9.5, although other pH values may be suitable for certain applications. The pH of the matrix material may be controlled by buffering the first and/or second components using conventional buffering materials such as phosphate, borate, and bicarbonate buffers.

The cure time of the composition can also be adjusted by changing the molecular weight of the B component (e.g. the amine reactive component). Typically, amine reactive components formed from lower molecular weight polyol components (as compared to higher molecular weight polyol components) favor high reactivity (i.e., shorter cure times). This can be accomplished, for example, by controlling the molecular weight of the hydroxyl-functional material that is used to form in the amine-reactive component.

The molecular weight and functionality of the B component (e.g., amine-reactive component) can also affect the physical properties of the matrix formed upon cure. As such, the B component can be chosen to provide a body member with particular physical properties. The present invention shows that B components having a lower molecular weight (such as lower molecular weight poly(ethylene glycols)) provide a matrix having an increase in one or more of density, and/or hardness. By contrast, as the molecular weight of the B component increases, the matrix material becomes softer and more flexible.

A similar observation can be made with respect to functionality. As the functionality of the B component increases, the matrix has an increase in one or more of density, and/or hardness.

The physical properties may be modified in order to achieve desired properties for a given end-use. For example, a shape memory prosthesis can be prepared from a mixture of the A and B components that provides a combination of harness and flexibility, and that enables the article to exert force on a tissue at the target site in the first configuration.

In some aspects, the coupling group on the hydrophilic polymer is a polymerizable group. In a free radical polymerization reaction, the polymerizable group can couple hydrophilic polymers together in the composition, thereby forming a hydrophilic polymer matrix, which forms the body member.

A preferred polymerizable group is an ethylenically unsaturated group. Suitable ethylenically unsaturated groups include vinyl groups, acrylate groups, methacrylate groups, ethacrylate groups, 2-phenyl acrylate groups, acrylamide groups, methacrylamide groups, itaconate groups, and styrene groups. Combinations of different ethylenically unsaturated groups can be present on a hydrophilic polymer.

Hydrophilic polymers can be effectively derivatized in an appropriate solvent system to produce macromers. Generally, a solvent system is used that allows for polymer solubility and control over the derivatization with polymerizable groups. A particularly useful solvent for polymer derivatization is formamide. Other solvents or solvent combinations may be used.

Macromer preparation (addition of polymerizable groups to the polymer) can be carried out using any suitable method. Polymerizable groups such as glycidyl acrylate can be added to a hydrophilic polymer, such as a polysaccharide, in straightforward synthetic processes.

For example, a polysaccharide can be reacted with a compound containing a polymerizable group, such as glycidyl acrylate, in the presence of formamide (and TEA, for pH control) to provide acrylate-derivatized polysaccharides. The number and/or density of acrylate groups can be controlled using the present method, e.g., by controlling the relative concentration of reactive group to saccharide group content.

In some modes of practice, the hydrophilic polymer have an amount of pendent coupling groups of about 0.7 μmoles of coupling group per milligram of hydrophilic polymer or less. In a preferred aspect, the amount of coupling group per hydrophilic polymer is in the range of about 0.2 μmoles/mg to about 0.7 μmoles/mg. For example, a hydrophilic polymer such as amylose or maltodextrin can be reacted with an acrylate group-containing compound to provide an amylose or maltodextrin macromer having a acrylate group load level in the range of about 0.3 μmoles/mg to about 0.7 μmoles/mg.

In some aspects, the body member comprises a matrix that is formed of an inter penetrating network (“IPN”) comprising hydrophilic polymeric components. An IPN is a matrix of at least two polymeric networks that penetrate, but that are not crosslinked to each other. The two polymeric networks can be partially or fully interpenetrating.

For purposes of discussion, an aspect of the invention wherein the body member is formed from an IPN that is formed from an “A” polymeric network and a “B” polymeric network is described. One or more additional polymeric networks (“C”, etc.) can be formed if desired.

Generally, in IPN formation, the polymeric networks are formed using different chemical coupling mechanisms (such as those described herein, including polymerization methods), so the individual networks do not become bonded to each other. For example, the A network can be formed using a free radical polymerizable components, and the B network can be formed using a components that are coupled via an nucleophilic reaction using first and second reactive groups. Alternatively, the body member can be formed by ionically crosslinking the hydrophilic polymers, such as by cationic or anionic polymerization. As another option, complex coordinative polymerization can be used.

In some aspects the body member is formed from an IPN including an A polymeric network formed from a first hydrophilic polymer and a B polymeric network formed from a second hydrophilic polymer that is the same or different than the first hydrophilic polymer. In preferred embodiments, both A and B polymeric networks are formed from hydrophilic biodegradable polymers, such as natural biodegradable polysaccharides. For example, the A and B polymeric networks can be formed of polysaccharides selected from the group consisting of amylose, maltodextrin, and polyalditol. Other hydrophilic components can be used to form the networks, if desired.

To exemplify formation of a biodegradable body member of the shape memory article, a polymeric network is formed from a biodegradable polysaccharide having a first reactive group “component A₁” (such as aminated polyalditol) a hydrophilic component having a second reactive group “component A₂” (such as an amine reactive polyethylene glycol). The B polymeric network is formed from a biodegradable polysaccharide having polymerizable groups “component B” (such as acrylated maltodextrin or acrylated polyalditol).

A composition can be prepared by preparing a first composition that includes components A₁ and B, which are generally non-reactive in combination. The composition can also include components useful for the free-radical polymerization reaction, such as an initiator, and one or more ancillary reagent(s) such as co-initiators, reducing agents, and/or polymerization accelerants. A second composition can include component A₂. Other components can be included in the second composition if desired (such as those that would promote free-radical polymerization reaction of component B).

The first and second compositions can be mixed, and immediately placed in a first configuration (such as in a mold). Following mixture, the pendent reactive groups of components A₁ and A₂ react to form a first polymeric network A₁₋₂, which can cause the composition to set up into the shape of the first configuration. In this state, the first network may impart some properties desirably associated with the shape memory article, such as flexibility. The mixture can then be treated to promote polymerization of component B, which forms a second network that penetrates the first network. For example, a photoinitiator can be present in the mixture, and the mixture can be treated to promote free-radical polymerization of component B. Formation of the second network can change the properties of the matrix, as compared to the formation of the first network alone. For example, formation of the second network may add properties such as strength to the shape memory article.

In some specific aspects, component A₁ comprises polyalditol having pendent first reactive groups, such as pendent amine groups; component A₂ comprises a hydrophilic alkoxyalkane of about 10,000 Da or less and having pendent amine-reactive groups; and component B comprises a natural biodegradable polysaccharide such as maltodextrin, polyalditol, or amylose, and having pendent polymerizable groups. Preferably, the polysaccharide components (A₁ and B) have a molecular weight of about 100,000 Da or less, about 50,000 Da or less, or preferably about 25,000 Da or less.

In preparing a composition for forming the shape memory article, the hydrophilic polymer is dissolved in a suitable liquid. Typically, the hydrophilic polymer is dissolved in water. Optionally, the hydrophilic polymer is dissolved in an aqueous solution containing a mixture of water and a water-miscible organic liquid. In some aspects an alcohol, such as ethanol, can be included in the composition containing the hydrophilic polymer.

The hydrophilic polymer is dissolved at a concentration in the composition sufficient to form a hydrophilic shape memory article. Depending on the desired use of the implantable article, a composition is prepared with a hydrophilic polymer at a predetermined concentration in order to provide the body member of the article with a suitable internal strength.

In some aspects, compositions having a relatively high concentration of hydrophilic polymer can be used to produce an insertable medical article having an internal strength sufficient to revert from a second configuration to a first configuration, wherein the second configuration is capable of performing a mechanical function in the body. For example, the article in the second configuration can be capable of exerting force on body tissue which can be of therapeutic benefit to a subject. In some aspects the article in the second configuration is an implantable prosthetic device, such as a stent. In this regard, the body member has internal strength not only to revert from a second configuration to a first configuration, but also to exert force upon a tissue in the first configuration. For example, in the first configuration, the body member can exert force upon the inner wall of a vessel. In the second configuration, the stent can be delivered through the vasculature to a target site.

The hydrophilic components are present in the compositions at concentrations for forming a body member to provide an article with shape memory properties. The compositions used to form the body member have a concentration of matrix-forming material of at least 50 mg/mL. In some modes of preparing the shape memory article, and more typically, a composition having a concentration of one or more matrix-forming components (e.g., the hydrophilic polymer) of about 250 mg/mL (20% wt. solids) or greater is prepared and used to form the body member. In preparations the concentration of the matrix-forming components can be 300 mg/mL (23% wt. solids) or greater, 500 mg/mL (33% wt. solids) or greater, or even 750 mg/mL (43% wt. solids) or greater. Natural biodegradable polysaccharides, such as one selected from amylose, maltodextrin, cyclodextrin, polyalditol, and/or hydrophilic polymers such as PEG can be used to form compositions with very high solids content. Such articles can therefore be prepared with a high degree of internal strength using these compositions.

Following its formation, and in a hydrated state, the body member has a percentage of matrix forming materials (e.g., one or more hydrophilic polymers) and water. Generally the body member has about 15% or greater of matrix-forming materials, such as in the range of about 15% to about 75% matrix-forming materials. Preferably, the body member comprises about 30% to about 50% matrix-forming materials. In some cases the body member has a total solids content of matrix forming components of about 40%. If the body member is dehydrated, the matrix forming materials as a percentage of the total weight of the body member, increases.

In other aspects, compositions having a lower concentration of hydrophilic polymer can be used to produce an insertable medical article with a shape memory property. In these aspects, the body member can have internal strength sufficient to revert from a second configuration to a first configuration. However, the body member may not be required to exert force upon a tissue at the target site such as to provide a prosthetic function at the target site. In these aspects, for example, the body member may revert to a first configuration that is particularly suitable for residence at the target site and can provide one more other non-prosthetic functions.

For example, the target site can be an area within the posterior portion of the eye. The shape memory article can revert from a second configuration to a first configuration upon insertion in the eyeball. In the first configuration the body member is non-linear and provides an improved article for delivering a bioactive agent to the eye. The first configuration also does not interfere with the central visual field because it is non-linear.

The target site can also be an aneurysm. The shape memory article can be delivered to the aneurysm, wherein the article reverts to a first configuration that substantially fills the aneurysm.

The body member can be prepared from a composition having a predetermined amount of liquid, such as water, present in the composition. In some aspects the composition comprises about 50 wt % liquid or less; in some aspects the composition comprises about 15 wt % liquid or less. Upon formation of the body member, the amount of liquid can be retained in the body member, or the amount can be changed, such as by removal of liquid from the body member.

Other components can be added to the composition. In some aspects other components are added to the composition as compounds that can promote formation of the body member upon treating the composition.

For example, biocompatible hydrophilic crosslinking components having unsaturated groups can also be included in the composition. For example, the composition can include crosslinking agents such as diallyl itaconate, diallyl maleate, diallyl fumarate, dimethallyl fumarate, dimethallyl maleate, diallyl diglycollate, diethylene glycol bis(allyl carbonate), diallyl oxalate, diallyl adipate, diallyl succinate, diallyl azelate, divinyl benzene, divinyl adipate, and divinylethers. The crosslinking agent, or a combination of crosslinking agents can be present in the composition in the amount of 1-40%, more preferably in the amount of 5-20%.

In some aspects, the composition includes an initiator. As used herein, an “initiator” refers to a compound, or more than one compound, that is capable of promoting the formation of a reactive species from a polymerizable group. For example, the initiator can promote a free radical reaction of hydrophilic polymer having a pendent polymerizable group. In one embodiment the initiator is a compound that includes a photoreactive group (photoinitiator). For example, the photoreactive group can include an aryl ketone photogroup selected from acetophenone, benzophenone, anthraquinone, anthrone, anthrone-like heterocycles, and derivatives thereof.

In some aspects the photoinitiator includes one or more charged groups. The presence of charged groups can increase the solubility of the photoinitiator (which can contain photoreactive groups such as aryl ketones) in an aqueous system. Suitable charged groups include, for example, salts of organic acids, such as sulfonate, phosphonate, carboxylate, and the like, and onium groups, such as quaternary ammonium, sulfonium, phosphonium, protonated amine, and the like. According to this embodiment, a suitable photoinitiator can include, for example, one or more aryl ketone photogroups selected from acetophenone, benzophenone, anthraquinone, anthrone, anthrone-like heterocycles, and derivatives thereof; and one or more charged groups. Examples of these types of water-soluble photoinitiators have been described in U.S. Pat. No. 6,077,698.

Thermally reactive initiators can also be used to promote the polymerization of hydrophilic polymers having pendent coupling groups. Examples of thermally reactive initiators include 4,4′ azobis(4-cyanopentanoic acid), 2,2-azobis[2-(2-imidazolin-2-yl) propane]dihydrochloride, and analogs of benzoyl peroxide. Redox initiators can also be used to promote the polymerization of the hydrophilic polymers having pendent coupling groups. In general, combinations of organic and inorganic oxidizers, and organic and inorganic reducing agents are used to generate radicals for polymerization. A description of redox initiation can be found in Principles of Polymerization, 2^{nd} Edition, Odian G., John Wiley and Sons, pgs 201-204, (1981).

The polymerization initiator can also be present on a hydrophilic polymer that includes an initiator group (herein referred to as an “initiator polymer”). The polymeric portion of the initiator polymer can be the same or different than that of one or more hydrophilic polymers that are used to form the body member.

The composition that includes a hydrophilic polymer with pendent polymerizable groups and a polymerization initiator can also include one or more other ancillary reagent(s) that help promote formation of the body member. These reagents can include polymerization co-initiators, reducing agents, and/or polymerization accelerants known in the art. These ancillary agents can be included in the composition at any useful concentration.

Exemplary co-initiators include organic peroxides, such as those that are derivatives of hydrogen peroxides (H₂O₂) in which one or both of the hydrogen atoms are replaced by an organic group. Organic peroxides contain the —O—O— bond within the molecular structure, and the chemical properties of the peroxides originate from this bond. In some aspects of the invention, the peroxide polymerization co-initiator is a stable organic peroxide, such as an alkyl hydroperoxide. Exemplary alkyl hydroperoxides include t-butyl hydroperoxide, p-diisopropylbenzene peroxide, cumene hydroperoxide, acetyl peroxide, t-amyl hydrogen peroxide, and cumyl hydrogen peroxide.

Other polymerization co-initiators include azo compounds such as 2-azobis(isobutyro-nitrile), ammonium persulfate, and potassium persulfate.

In some aspects of the invention, the composition used to form the body member can include a reducing agent such as a tertiary amine. In many cases the reducing agent, such as a tertiary amine, can improve free radical generation. Examples of the amine compound include primary amines such as n-butylamine; secondary amines such as diphenylamine; aliphatic tertiary amines such as triethylamine; and aromatic tertiary amines such as p-dimethylaminobenzoic acid.

In other aspects of the invention, in addition to these components, the composition used to form the body member can include one or more polymerization accelerator(s). Polymerization accelerators such as n-vinyl pyrrolidone can be used. In some aspects a polymerization accelerator having a biocompatible functional group (e.g., a biocompatible polymerization accelerator) is included in the composition of the present invention. The biocompatible polymerization accelerator can also include an N-vinyl group such as N-vinyl amide group. Biocompatible polymerization accelerators are described in commonly assigned U.S. Patent Application Publication No. 2005/0112086.

In some aspects of the invention, the article provides a mechanical feature to a target portion of the body when the body member is in a first configuration. In the first configuration the body member can exert force on tissue at the target location. In this regard, this mechanical feature may obviate the need for one or more other forms of treatment at the target location, such as treatment using a drug.

In some aspects, a bioactive agent can be associated with the body member of the hydrophilic shape memory article. In some aspects, the hydrophilic shape memory article is primarily used to delivery a bioactive agent to a subject, such at the target location. In this regard, the hydrophilic shape memory article may serve primarily as a drug delivery device and can provide a local pharmacological activity at the target location.

In other aspects, a bioactive agent can be associated with a hydrophilic shape memory article that provides a mechanical feature to the target location when the article is in the first configuration. The bioactive agent can be chosen to provide a therapeutic effect that may supplement the mechanical feature provided by the article.

If a bioactive agent is associated with the body member, it can be associated with the body member in any suitable manner. For example, the bioactive agent can be released from the article, or can be stably associated with the surface of the article.

In some aspects of the invention a bioactive agent can be included in the body member of the shape memory article. For example, the bioactive agent can be present in the body member formed from hydrophilic biodegradable polymers and can be released from the body member as it degrades. The body member can therefore serve as a medium for the slow or controlled release of the bioactive agent.

The bioactive agent can also be present in a coating formed on the body member. The coating can be a biodegradable or biostable coating. In some aspects the bioactive agent is present in a biostable or biodegradable body member comprising a biodegradable coating. A biodegradable coating can be formed using the natural biodegradable polysaccharides as described herein.

A bioactive agent can also be included in or as microparticles that are associated with the body member. For example, the body member can include microparticles having a bioactive agent that can be released from the body member. In some aspects, the body member includes microparticles comprising a low molecular weight bioactive agent. The use of microparticles can represent one method of controlling the release of low molecular weight bioactive agent form the body member of the shape memory article. Examples of microparticle systems useful for delivering a low molecular weight bioactive agent are describe in Applicants' U.S. Provisional Application No. 60/759,241, filed Jan. 13, 2006.

The term “bioactive agent,” refers to an inorganic or organic molecule, which can be synthetic or natural, that causes a biological effect when administered in vivo to an animal, including but not limited to birds and mammals, including humans.

In some aspects, the shape memory articles prepared according to the invention can be used to release bioactive agents falling within one or more of the following classes include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, and vasospasm inhibitors.

In other aspects the shape memory article can include a high molecular weight bioactive agent. Although not limited to such, the body members of the invention are particularly useful for delivering bioactive agents that are high molecular weight bioactive agents, such as polypeptides (including proteins and peptides), nucleic acids (including DNA and RNA), and polysaccharides (including heparin). The bioactive agent can be directly mixed with a composition that is used to form the body member of the article. Alternatively the high molecular weight bioactive agents can be present in or as microparticles. In some cases the bioactive agent has a molecular weight of 10,000 or greater.

In some cases the high molecular weight bioactive agent is a therapeutic antibody or fragments thereof. Examples of these agents include, trastuzumab (Herceptin™), a humanized anti-HER2 monoclonal antibody (moAb); alemtuzumab (Campath™), a humanized anti-CD52 moAb; gemtuzumab (Mylotarg™), a humanized anti-CD33 moAb; rituximab (Rituxan™), a chimeric anti-CD20 moAb; ibritumomab (Zevalin™), a murine moAb conjugated to a beta-emitting radioisotope; tositumomab (Bexxar™), a murine anti-CD20 moAb; edrecolomab (Panorex™), a murine anti-epithelial cell adhesion molecule moAb; cetuximab (Erbitux™), a chimeric anti-EGFR moAb; and bevacizumab (Avastin™), a humanized anti-VEGF moAb.

In some aspects the bioactive agent can be selected to improve the compatibility (for example, with blood and/or surrounding tissues) of the surface of the shape memory article. These agents, referred to herein as “biocompatible agents,” when associated with the shape memory article, can reduce the likelihood for blood components to adhere to the medical article, thus reducing the formation of thrombus or emboli (blood clots that release and travel downstream).

Representative examples of bioactive agents having antithrombotic effects include heparin, heparin derivatives, sodium heparin, low molecular weight heparin, hirudin, lysine, prostaglandins, argatroban, forskolin, vapiprost, prostacyclin and prostacyclin analogs, D-phenylalanyl-L-prolyl-L-arginyl-chloromethylketone (synthetic antithrombin), dipyridamole, glycoprotein IIb/IIIa platelet membrane receptor antibody, coprotein IIb/IIIa platelet membrane receptor antibody, recombinant hirudin, thrombin inhibitor (such as commercially available from Biogen), chondroitin sulfate, modified dextran, albumin, streptokinase, tissue plasminogen activator (TPA), urokinase, nitric oxide inhibitors, and the like.

The bioactive agent can also provide antirestenotic effects, such as antiproliferative, anti-platelet, and/or antithrombotic effects. In some embodiments, the bioactive agent can include an anti-inflammatory agent, immunosuppressive agent, cell attachment factor, receptor, ligand, growth factor, antibiotic, enzyme, nucleic acid, and the like. Compounds having antiproliferative effects include, for example, actinomycin D, rapamycin, analogues of rapamycin, angiopeptin, c-myc antisense, paclitaxel, taxane, 13-cis retinoic acid, retinoic acid derivatives, 5-fluorouracil.

In some aspects the bioactive agent is an anti-inflammatory agent such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, or triamcinolone acetonide. In some aspects the bioactive agent is an inhibitor of angiogenesis such as anecortave acetate or a receptor tyrosine kinase antagonist.

In some aspects, the bioactive agent can include polymerizable groups and can be formed into the body member of the shape memory article. For example, a polymeric bioactive agent, such as a polysaccharide, a polypeptide, or a polynucleotide, including polymerizable groups can be included in the composition and polymerized along with the hydrophilic polymer. The bioactive agent can be stably or releasably incorporated into the body member.

For example, the body member can be formed from hydrophilic biodegradable polymer such as a natural biodegradable polysaccharide having a pendent polymerizable group and a bioactive polymer, or an active portion thereof, having a pendent polymerizable group. This can provide a biodegradable body member that is capable of releasing a bioactive polymer, or an active portion thereof, upon degradation of the body member.

In some cases, the body member can include a bioactive agent in the form a pro-fibrotic macromer, such as a collagen macromer. A body member formed with a pro-fibrotic macromer can be particularly useful in the preparation of occlusion devices, such as devices that are useful for the treatment of aneurysms. For example, a shape memory aneurysm device having a biodegradable body member that can assume a first coiled configuration can be used fill an aneurysm. Degradation of the body member causes release of collagen and promotes a thrombotic response in the vicinity of the body member, thereby providing an improved system for occluding the aneurysm. A pro-fibrotic macromer can also be used to provide a polymeric network in an IPN that is used to form the body member.

Polymerizable groups can be added to collagen, or an active portion thereof, via reaction of amine containing lysine residues with acryloyl chloride. Collagen can be dissolved in formamide with the addition of acryloyl chloride (and TEA, for pH control) to provide acrylate-derivatized collagen molecules.

In some cases, the body member can include a bioactive agent in the form an anti-thrombotic macromer, such as a heparin macromer. A body member formed with an anti-thrombotic macromer can be particularly useful in the preparation of stents. For example, a shape memory stent having a body member that can assume a first expanded configuration can be used at an intravascular target site. The stent provides a mechanically useful function at the target site for a predetermined amount of time (i.e., the in vivo lifetime of the stent as based on the rate of degradation of the stent) and also prevents thrombus formation by releasing an anti-thrombotic agent, such as heparin, in the vicinity of the stent. A heparin macromer can be prepared by various techniques, such as by reaction with glycidyl acrylate as described herein. An anti-thrombotic macromer can also be used to provide a polymeric network in an IPN that is used to form the body member.

A radiopacifying agent can also be included in a composition that is used to form the body member. The radiopacifying agent can improve imagining of the shape memory article that is inserted within the body. This can improve detection of the shape memory article during and/or after insertion to a desired location in the body.

In some specific aspects, the radiopacifying agent comprises iodine. Iodine can be used in conjunction with a biodegradable shape memory body member formed from a natural biodegradable polysaccharide. Natural biodegradable polysaccharides have been found to complex iodine, thereby providing a useful way of improving the imaging of an article in the body. Release of iodine during or after degradation of the body member formed from the polysaccharide is non-toxic.

The radiopacifying agent can be iodine, or a secondary compound, such as a commercially available iodine-containing radiopacifying agent.

The radiopacifying agent can also be a radioisotope, such as 1125. The radioisotope may also serve a secondary function, such as the radiotherapeutic treatment of tissue that is in contact with the body member of the shape memory article.

In some cases, the shape memory article is introduced temporarily or permanently into a mammal for the prophylaxis or treatment of a medical condition. The shape memory article can be introduced subcutaneously, percutaneously or surgically to rest within an organ, tissue, or lumen of an organ, such as arteries, veins, ventricles, or atria of the heart.

Exemplary medical articles include vascular implants and grafts, stents, surgical devices; synthetic prostheses; vascular prosthesis including endoprosthesis, stent-graft, and endovascular-stent combinations; small diameter grafts, abdominal aortic aneurysm grafts; anastomosis devices and anastomotic closures; aneurysm exclusion devices; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; orthopedic devices such as orthopedic joint implants, bone repair/augmentation devices, cartilage repair devices; urological devices and urethral devices such as urological implants, bladder devices, and renal devices and hemodialysis devices.

In some particular aspects, the shape-memory article is used as a prosthesis in the cardiovascular or urogenital systems.

Articles configured for placement at an internal site of the eye can reside within any desired area of the eye. In some aspects, the shape memory article is an ophthalmic article configured for placement at an intraocular site, such as the vitreous. The shape memory article can have a first configuration that is based on the devices described in U.S. Pat. Nos. 6,719,750 B2 (“Devices for Intraocular Drug Delivery,” Varner et al.) and 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and U.S. application Ser. Nos. 11/204,195 (filed Aug. 15, 2005, Anderson et al.), 11/204,271 (filed Aug. 15, 2005, Anderson et al.), 11/203,981 (filed Aug. 15, 2005, Anderson et al.), 11/203,879 (filed Aug. 15, 2005, Anderson et al.), 11/203,931 (filed Aug. 15, 2005, Anderson et al.); and related applications.

Shape memory implantable articles of the invention can have simple or complex geometries. A simple geometry is exemplified by a medical article that is in the form of a filament (e.g., threads, strings, rods, etc.). The filament can have a first configuration, in which the article is formed, and which is the “memory” configuration. In the case of a filament, the first configuration can be non-linear, for example, wherein the filament has a curved configuration, a coiled configuration, a bent configuration, etc. The second configuration can be any configuration that is different than that of the first configuration. For example, if the first configuration is a coil configuration having particular dimensions and shape (e.g., an outer diameter, an inner diameter, coil spacing, curvature, etc.) the second configuration can be any configuration that is different than the particular dimensions of the first configuration.

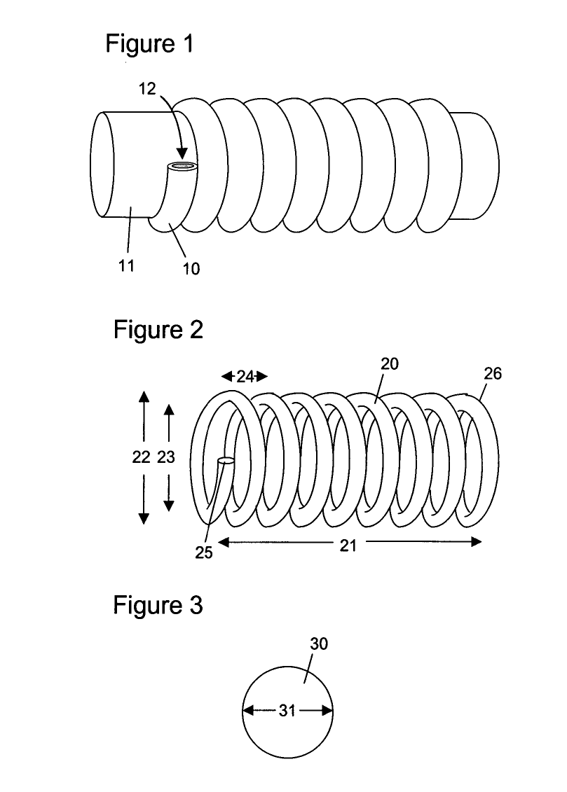

A shape memory article with a simple geometry can be prepared by various methods. One method for preparing the shape memory article having a simple geometry, such as a filament, can include a step of forming body member in the first configuration in a mold. In one mode of practice, a composition comprising a hydrophilic polymer having pendent polymerizable groups and a polymerization initiator is disposed in a mold. The mold can be, for example, flexible tubing fixtured in a certain configuration that corresponds to a first configuration of the body member. For example, as shown in FIG. 1, a mold is formed by wrapping flexible tubing 10 around a mandrel 11 in a coil configuration, wherein the tubing is fixtured to stabilize the configuration. Alternatively, the mold can be non-flexible.

A composition 12 including the matrix-forming components can be injected into the tubing to fill the tubing. The composition can be treated to activate the polymerization initiator (such as by photo-initiated or thermally initiated polymerization). Polymerization promotes crosslinking of the hydrophilic polymers and establishes a polymeric matrix in this first configuration. The polymeric matrix provides an internal strength to the body member, so the body member has a memory for this first configuration. That is, when the body member is reconfigured into a second configuration, which can be any configuration other than the first configuration, the internal strength of the body member allows the body member to revert from the second configuration to the first configuration.

In some modes of preparation, a composition comprising a reactive hydrophilic polymer comprising a first reactive group, and a second component that is hydrophilic and that comprises a second reactive group is used to form the body member. Since the polymer and the second component react upon mixture, it is generally desirable to immediately deliver the mixture to a mold following mixing. Alternatively, the polymer and the second component are independently delivered to a mold, in which they are mixed.

In an exemplary embodiment, the first and second components are held in separate chambers of mixing device, such as a dual syringe mixing device. When cure of the matrix is desired, simultaneous application of hand pressure to both syringe plungers in the device causes both the first and second component to flow from their respective syringes into a stationary mixing device (e.g., a “split flow” type mixer) where the first and second components are mixed with one another at a predetermined ratio. After being mixed, the matrix-forming composition exits the device though a single outlet orifice and into a mold which is in the first configuration. Useful dual syringe mixing devices are commercially available under the trade designation “MIXPAC” from Mixpac Systems AG (Rotkreuz, CH).

In order to promote efficient mixing, it is generally desirable for the first component and the second component to be formulated to have similar viscosities. In many embodiments, the first and the second component have viscosities up to about 500 cps.

In some aspects, after the body member is formed in the first configuration it can be removed from the mold. The body member can be forced out of the mold using hydrostatic pressure, or the mold can be broken to remove the body member. As shown in FIG. 2, the body member emerges from the mold in the first configuration 20.

Referring again to FIG. 2, the body member in the first configuration 20, which is a coil configuration, has certain dimensions. The body member in the first configuration 20 has a length 21, an outer diameter 22, an inner diameter 23, and a spacing 24. The body member in the first configuration also has a first end 25 and a second end 26.

FIG. 3 shows a cross section of the filament 30 that is formed by the mold, and shows the shape of the cross section, which in this aspect is circular. The circular cross section has a diameter 31. While the filament is shown having a cross sectional shape that is circular, the filament can have any cross sectional shape, as dictated by the shape of the mold. For example, the cross section of the filament can have any curved cross sectional shape, such as a circular or oval cross sectional shapes. The cross sectional shape can also include a straight portion, including any polygonal shape, such as triangular, square, rectangular, hexagonal, octagonal, etc.

The cross sectional of the article can also defined by a cross-sectional area, which can, in many aspects, be very small. For example, in some cases, the cross-sectional area of the article can be about 1.5 mm² or less, about 1.0 mm² or less, or even about 0.5 mm² or less. The small cross sectional area can facilitate delivery of the article to a target site in the body.

Prior to implantation into the body, the body member of the shape memory article is reconfigured into a second configuration. The second configuration can be of any configuration that is different than the first configuration. In some aspects, the body member in the first configuration is reconfigured into a second configuration and held in the second configuration by an insertion instrument that is used to deliver the shape memory article to a target location in the body. For example, the body member is held in the second configuration by a needle or catheter. In this aspect, the body member can be kept in a hydrated state or, optionally, but not required, a dehydrated state. If the body member is in a hydrated state, the insertion instrument prevents the body member from reverting from the second configuration to the first configuration.

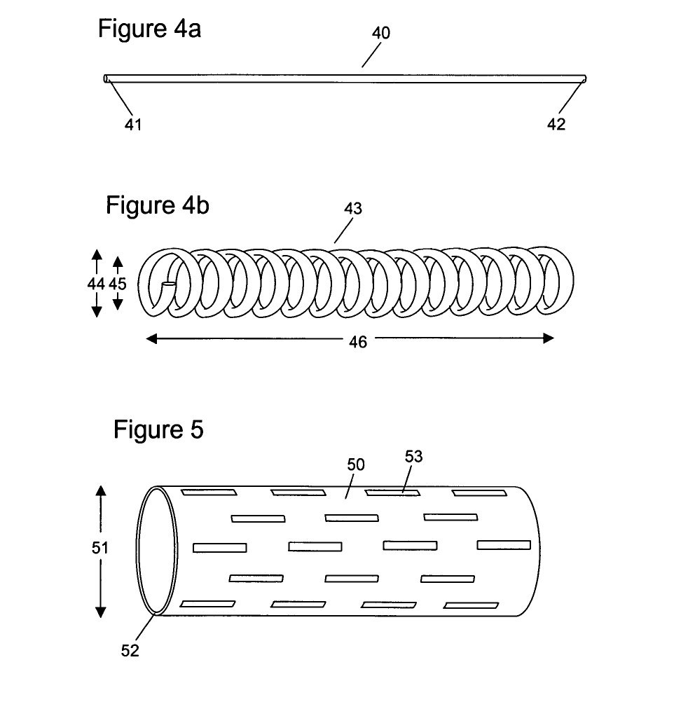

As stated, the body member can be reconfigured into any suitable second configuration. For example, the body member with the coiled first configuration as shown in FIG. 2 is reconfigured into a second configuration 40 that is linear, as shown in FIG. 4a. The second configuration 40 is that of an extended coil. The body member in the second configuration has a first end 41 and a second end 42. The distance along the body member between the first end 41 and the second end 42 of the second configuration will be will be generally the same as the distance of the body member between first end 25 and the second end 26 of the first configuration following the path of the coil. However, the length of the body member in the second configuration will be greater than the length 21 of the body member in the first configuration.

Another example of a second configuration is a coil configuration that is different than the coil in the first configuration. For example, as shown in FIG. 4b, the second configuration 43 is that of a coil that is more tightly wound than the coil shown in FIG. 2. The second configuration 43 has an outer diameter 44 and an inner diameter 45 that is less than the outer diameter 22 and an inner diameter 23, respectively, of the coil in the first configuration 20. In turn, the length 46 of the body member in the second configuration can be greater than the length 21 of the body member in the first configuration. Alternatively, the spacing between the filaments in the second configuration may be less than the spacing 24 between the filaments in the first configuration (not shown).

In other aspects of the invention, the body member is reconfigured from the first configuration to a second configuration, and then the body member is dehydrated in the second configuration to stabilize the configuration of the body member. In the dehydrated configuration the body member is required to be stabilized by a secondary device, such as a needle or a catheter. Upon rehydration, the body member reverts from the second configuration to the first configuration (referred to herein as hydration state-based shape memory).

In order to stabilize the second configuration of the article in a dehydrated state, following the step of forming the body member, the body member is reconfigured into a second configuration, held in that configuration, and then dehydrated while the body member is held in the second configuration. In some modes of practice, if a flexible mold is used, the mold can be reconfigured to the second configuration and the body member can be dehydrated while in the mold. After the body member is dehydrated it can be removed from the mold, for example, by cutting away the mold from the body member.

Dehydration can be carried out using any suitable technique. For example, the body member can be heated or placed in a reduced humidity environment. The body member is dehydrated for a period of time sufficient for removal of an amount of water from the body member so the body member is maintained in the second configuration. The amount of water that is removed from the body member during the dehydration process may depend on the amount of water in the article prior to the dehydration process. For example, in some aspects, the body member in the first configuration has about 30% by weight water. The body member is reconfigured to the second configuration and about 50% or greater of water is removed from the body member, leaving the body member with not greater than 15% by weight water. In some cases about 50% to about 75% of the liquid present in the composition is removed. The amount of water removed from the body member during dehydration may depend on one or more factors, including the amount of water in the body member in the first configuration, the type(s) of hydrophilic polymer used to form the body member, the extent of crosslinking in the matrix of the body member, etc.

The dehydrated body member will have a cross sectional shape that is generally the same shape as in the hydrated form. That is, referring to FIG. 3, a filament will have the same circular cross sectional shape in both the hydrated and dehydrated states. However, the diameter of the filament in the dehydrated state will be somewhat less than the diameter in the hydrated state. Generally, this reduction in diameter in the dehydrated state will not be greater than about 10% or 15% of the diameter in the hydrated state using articles formed from compositions having a high concentration of hydrophilic polymer.

In another aspect, and referring to FIG. 5, the shape memory article has a first configuration in the shape of a cylinder 50. The cylinder 50 can have a size suitable for use within a vessel in the body. For example, in a first configuration the cylinder can have an outer diameter 51 of about 4 mm or less. In some aspects, the cylinder 50 is prepared for use as a stent. In the first configuration, the outer surface of the body member can exert force upon the inner wall of a vessel.

In some cases, also referring to FIG. 5, the cylinder can be formed or processed to include one or more fenestration 53. The fenestrations 53 can improve function of the cylinder within the body, such as by allowing the movement of fluid and cells through the fenestrations. The fenestrations can also provide improved structural features to the cylinder. The fenestrations can be formed as slits in the wall of the cylinder, or holes of any desired size or shape.

The cylinder 50 can be formed by disposing a composition in a mold and treating the composition to form the body member in a first configuration. This process may be similar to the process used to form a filament. A mold useful for the formation of a shape memory cylinder can be formed using a “tube in tube” approach, wherein a tube with a smaller diameter is placed within the inner diameter of a larger tube. The outer diameter of the smaller tube can be separated from the inner diameter of the larger tube using a spacer to provide a gap that is generally uniform around the circumference. A set of tubes with the appropriate spacers can be assembled to provide mold having a gap useful for forming a shape memory cylinder with a wall of a desired thickness. For example, using this method, an article having a wall with a thickness of about 2.5 mm or less, about 2.25 mm or less, or about 2.0 mm or less can be formed. In some cases the wall of the article has a thickness in the range of about 0.5 mm to about 2.0 mm, or about 1.0 mm to about 2.0 mm.

Alternatively, the cylinder 50 can be formed by disposing a composition on a mandrel and then treating the disposed composition to form the body member. A very thin-walled cylinder can be formed using this approach. For example, a composition including a hydrophilic macromer and a polymerization initiator can be dip-coated or spray coated on a mandrel and then treated to crosslink the macromers. Following treatment, a cylinder 50 with a particular wall thickness 52 can be obtained. Steps of disposing and treating can be repeated to increase the wall thickness 52. For example, a suitable wall thickness can be in the range of about 50 μm to about 150 μm.

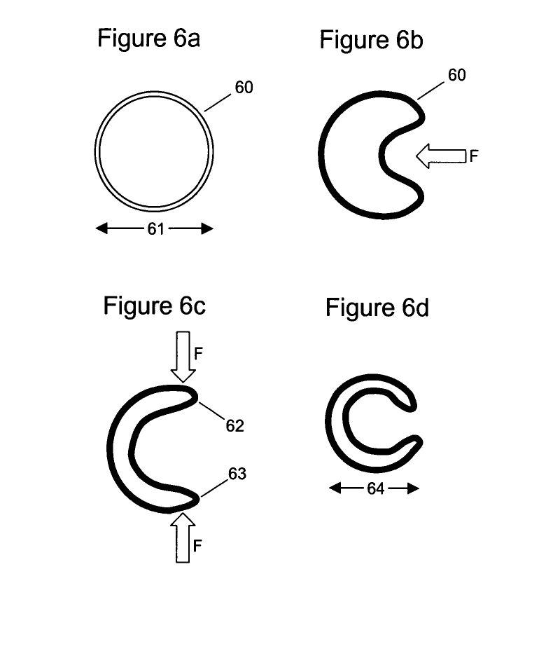

Prior to implantation into the body, the cylinder is reconfigured into a second configuration. Referring to FIG. 6a, a cross section of a cylinder 60 in a first configuration (fully expanded) state is shown, with outer diameter 61. The cylinder can be collapsed by applying force (F) (denoted by arrow F in FIG. 6b) along the outer wall of the cylinder at one (or more) location(s). This forces the cylinder into a “C” shaped configuration as shown in FIG. 6c. The collapsed cylinder can be further manipulated to reduce its outer diameter by applying force to bring points 62 and 63 closer to each other to provide the cylinder in a collapsed configuration as shown in FIG. 6d. The outer diameter 64 of the cylinder in this collapsed configuration is significantly less than the outer diameter 61 in the first configuration. This provides an advantage at least in the process of delivering the cylinder to a target site.

In some cases, the cylinder in the second configuration is dehydrated to stabilize the second configuration. In other cases, the cylinder is in a hydrated state and the second configuration is maintained by holding the cylinder in a insertion instrument, such as a catheter.

The cylinder in a collapsed configuration (for example, as shown in FIG. 6d) can be passed though a vessel to a target site, or can be placed within a catheter for delivery to a target site. Upon delivery to the target site, the cylinder reverts from the second (collapsed) configuration to the first (expanded) configuration. The cylinder in the first configuration can exert force upon the inner wall of a body vessel and provide a therapeutic effect to a subject.

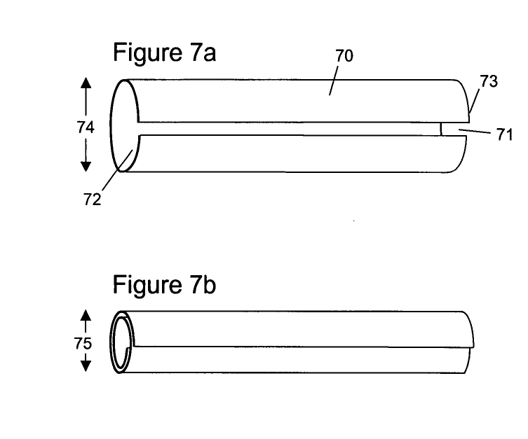

In another aspect, and referring to FIG. 7a, a cylinder 70 can be formed or processed in a first configuration to include a slit 71 running from the first end 72 to the second end 73. In the first configuration the cylinder 70 has an outer diameter 74 This allows the body member to be in the form of a rolled sheet in the first configuration exert force upon the inner wall of a body vessel. The cylinder body member can be reconfigured to a second configuration having a rolled shape as shown in FIG. 7b to reduce its outer diameter 75. The cylinder in the rolled configuration (for example, as shown in FIG. 7b) can be passed though a vessel to a target site, or can be placed within a catheter for delivery to a target site. Upon delivery to the target site, the cylinder reverts from the second (rolled) configuration to the first (expanded) configuration.

The shape memory article can also be provided as a system for the introduction of the article into a subject. The system (e.g., an implant delivery kit) includes, in the least, the shape memory article and an insertion instrument that facilitates the introduction of the shape memory article into a portion of the body. In the system, such as one delivered to a user such as a physician, the shape memory article can be loaded in a portion of the insertion instrument (such as in a needle of the insertion instrument), or can be provided separate.

If the article is loaded in a portion of the insertion instrument, it can be loaded in a second configuration. In some arrangements, the insertion instrument may constrain the article so that it is maintained in the second configuration. For example, the article in the first configuration can be generally linear, and the insertion instrument can include an article-retaining portion, such as a needle or catheter, that maintains the article in the second configuration. Alternatively, the article may be in the second configuration in a dehydrated form, and loaded into a portion of the insertion instrument. In this case, the instrument can be useful for delivering the article to a target site, but may not necessarily constrain the article in the second configuration.

During use, the article can be forced out of the article-retaining portion of the insertion instrument upon placing the distal end of the insertion instrument at a target location in the body. The article can be forced out using a mechanical feature of the insertion instrument, or by air or liquid pressure, or combinations of these. During of after the shape memory article exits the insertion instrument it reverts to the first configuration.

In a simple form the system can include as the insertion instrument a needle and plunger, or a needle and syringe, wherein the shape memory article is loaded in the needle. The insertion instrument can also be a catheter for delivery of a shape memory article that is an intravascular prosthesis.

The invention is also described with reference to the following non-limiting examples.

### EXAMPLE 1

### Preparation of Maltodextrin-Methacrylate Macromer (MD-Methacrylate)

To provide MD-methacrylate, the following procedure was performed. Maltodextrin (MD; Aldrich; 100 g; 3.67 mmole; DE: 4.0-7.0) was dissolved in dimethylsulfoxide (DMSO) 1,000 mL with stirring. The size of the maltodextrin was calculated to be in the range of 2,000 Da-4,000 Da. Once the reaction solution was complete, 1-methylimidazole (Aldrich; 2.0 g, 1.9 mL) followed by methacrylic-anhydride (Aldrich; 38.5 g) were added with stirring. The reaction mixture was stirred for one hour at room temperature. After this time, the reaction mixture was quenched with water and dialyzed against DI water using 1,000 MWCO dialysis tubing. The MD-methacrylate was isolated via lyophylization to give 63.283 g (63% yield). The calculated methacrylate load of macromer was 0.33 μmoles/mg of polymer

### EXAMPLE 2

### Synthesis of Aminated Polyalditol

Vacuum oven-dried Polyalditol PD60 (10.00 g) was dissolved with anhydrous dimethyl sulfoxide, DMSO, (50 mL) in a 120 mL amber vial. In a separate 30 mL amber vial, 1,1′-carbonyldiimidazole, CDI, (3.00 g) was dissolved in dry DMSO (25 mL). The CDI solution was poured into the maltodextrin solution and purged with nitrogen gas before being capped. The reaction solution was placed on a rotary shaker for 20 minutes. Into a separate 120 mL amber vial, 1,6-diaminohexane (10.80 g) was warmed to 45° C. and dissolved in dry DMSO (10 mL) and a Teflon stir bar was inserted and placed on a stir plate. The maltodextrin/CDI solution was slowly poured into the stirred diamine solution after 20 minutes. Once the addition was complete the reaction vial was transferred into a 55° C. oven and allowed to stir overnight. The next day, the reaction solution was precipitated into 1 liter tetrahydrofuran, THF, and a white precipitate formed. The mixture was stirred for one hour and the solvent was decanted. Fresh THF (1 L) was poured into the 2-L Erlenmeyer beaker and the white precipitate was stirred for one hour. This step was repeated twice. The final mixture was filtered using a water-aspirator, Büchner funnel, and Whatman-brand paper filter and a white precipitate was collected (13.14 g). The precipitate was then dried overnight at 40° C. under vacuum. A small sample of the material (50 mg) was dissolved with 5 mL deionized water in a 7-mL vial. To this sample was added 1 mL of ninhydrin solution (3.6 mg/mL in IPA). The sample was capped and heated to 70° C. in a water bath for a couple minutes, after which time the solution turned a dark purple color indicating the presence of primary amines.

### EXAMPLE 3

### Poly(ethylene glycol)₃₃₅₀-di(imidazolyl carbamate)

Vacuum oven-dried poly(ethylene glycol), MW˜3350, (6.70 g) was dissolved with anhydrous tetrahydrofuran, THF, (20 mL) in a 60 mL amber vial with slight heating (40° C.). In another 60 mL amber vial 1,1′-carbonyldiimidazole, CDI, (0.811 g) was dissolved with 10 mL dry THF. A Teflon stir bar was inserted into the CDI solution and placed on a stir plate. The PEG solution was pipetted into the CDI solution while stirring at room temperature. The reaction vial was purged with nitrogen gas once the addition was complete. The reaction was allowed to stir at room temperature for two hours. After two hours, the reaction solution was precipitated into 1 liter of chilled, anhydrous diethyl ether while stirring. The ether solution was decanted, and the precipitate was rinsed three more times (3×1 L) with fresh, anhydrous ether while stirring. The precipitate was collected by vacuum filtration using a water-aspirator, Büchner funnel, and a Whatman-type paper filter. The collected white precipitate (6.84 g) was dried overnight in a vacuum oven (30° C.).

### EXAMPLE 4

### Poly(ethylene glycol)₂₀₀₀-di(imidazolyl carbamate)

Vacuum oven-dried poly(ethylene glycol), MW 2000, (20.00 g) was dissolved with anhydrous tetrahydrofuran, THF, (200 mL) in a 500 mL amber vial with slight heating (40° C.). In another 500 mL amber vial 1,1′-carbonyldiimidazole, CDI, (4.10 g) was dissolved with 50 mL dry THF. A Teflon stir bar was inserted into the CDI solution and placed on a stir plate. The PEG solution was pipetted into the CDI solution while stirring at room temperature. The reaction vial was purged with nitrogen gas once the addition was complete. The reaction was allowed to stir at room temperature for two hours. After two hours, the reaction solution was precipitated into 2 liters of chilled, anhydrous diethyl ether while stirring. The ether solution was decanted and the precipitate rinsed three more times (3×1 L) with fresh, anhydrous ether while stirring. The precipitate was collected by vacuum filtration using a water-aspirator, Büchner funnel, and a Whatman-type paper filter. The collected white precipitate (19.41 g) was dried overnight in a vacuum oven (30° C.).

### EXAMPLE 5

### Poly(ethylene glycol)₁₅₀₀-di(imidazolyl carbamate)

Vacuum oven-dried poly(ethylene glycol), MW 1500, (15.00 g) was dissolved with anhydrous tetrahydrofuran, THF, (150 mL) in a 500 mL amber vial with slight heating (40° C.). In another 500 mL amber vial 1,1′-carbonyldiimidazole, CDI, (4.10 g) was dissolved with 50 mL dry THF. A Teflon stir bar was inserted into the CDI solution and placed on a stir plate. The PEG solution was pipetted into the CDI solution while stirring at room temperature. The reaction vial was purged with nitrogen gas once the addition was complete and the reaction was allowed to stir at room temperature for two hours. After two hours, the reaction solution was precipitated into 2 liters of chilled, anhydrous diethyl ether while stirring. The ether solution was decanted and the precipitate rinsed three more times (3×1 L) with fresh, anhydrous ether while stirring. The precipitate was collected by vacuum filtration using a water-aspirator, Büchner funnel, and a Whatman-type paper filter. The collected white precipitate (14.68 g) was dried overnight in a vacuum oven (30° C.).

### EXAMPLE 6

### Poly(ethylene glycol)₁₀₀₀-di(imidazolyl carbamate)

Poly(ethylene glycol), MW 1000, (20.59 g) was dissolved with anhydrous tetrahydrofuran, THF, (200 mL) in a 500 mL amber vial. In a 500 mL amber vial 1,1′-carbonyldiimidazole, CDI, (8.40 g) was dissolved with 50 mL dry THF. A Teflon stir bar was inserted into the CDI solution and placed on a stir plate. The PEG solution was pipetted into the CDI solution while stirring at room temperature. The reaction vial was purged with nitrogen gas once the addition was complete. The reaction was allowed to stir at room temperature for two hours. After two hours, the reaction solution was precipitated into 2 liters of chilled, anhydrous diethyl ether while stirring. The ether solution was decanted and the precipitate was rinsed three more times (3×1 L) with fresh, anhydrous ether while stirring. The precipitate was collected by vacuum filtration using a water-aspirator, Büchner funnel, and a Whatman-type paper filter. The waxy precipitate (17.59 g) was dried overnight in a vacuum oven (22° C.).

### EXAMPLE 7

### Poly(ethylene glycol)₆₀₀-di(imidazolyl carbamate)

Poly(ethylene glycol), MW 600, (30.15 g) was transferred to a 150 mL round bottom flask and dissolved with 50 mL dichloromethane (DCM). The solvent was stripped off using a rotary evaporator and high temperature water bath. This step was repeated twice more. In a 500 mL round bottom flask 1,1′-carbonyldiimidazole, CDI, (22.90 g) was dissolved with 250 mL DCM. A Teflon stir bar was inserted into the CDI solution and placed on a stir plate under nitrogen. The PEG₆₀₀ was dissolved with 50 mL DCM and slowly added to the stirring CDI solution and stirred at room temperature for two hours under nitrogen. The reaction solution was transferred into a 1 L separatory funnel and washed twice with 1 mM HCl followed by two brine solution washes. The organic solution was collected and dried with magnesium sulfate. The dried solution was filtered through a Whatman paper filter into a clean 500 mL round bottom flask and the DCM was rotary evaporated with mild heat (30° C.). A clear, slightly yellowish-tinted oil was collected (37.02 g).

### EXAMPLE 8

### Tetraethylene glycol-di(imidazolyl carbamate)

Tetraethylene glycol, TEG, MW 194.23, (21.80 g) was transferred to a 500 mL round bottom flask and dissolved with dichloromethane, DCM, (100 mL). The solvent was stripped off using a rotary evaporator and high temperature water bath. The stripping step was repeated twice. In a 1000 mL round bottom flask 1,1′-carbonyldiimidazole, CDI, (40.05 g) was dissolved with 380 mL DCM. A Teflon stir bar was inserted into the CDI solution and placed on a stir plate under nitrogen. The TEG was dissolved with 200 mL DCM and was slowly added to the stirred CDI solution, and the mixture was stirred at room temperature for two hours under nitrogen. The reaction solution was transferred into a 1 L separatory funnel and washed twice with 1 mM HCl followed by two brine solution washes. The organic solution was collected and dried with magnesium sulfate. The dried solution was filtered through a Whatman paper filter into a clean 500 mL round bottom flask and the DCM was rotary evaporated with mild heat (30° C.). A clear oil was collected (39.46 g).

### EXAMPLE 9

### Triethylene glycol-di(imidazolyl carbamate)

Triethylene glycol, TrEG, MW 150.17, (3.01 g) was transferred to a 50 mL round bottom flask and dissolved with dichloromethane, DCM, (30 mL). The solvent was stripped off using a rotary evaporator and high temperature water bath. The stripping step was repeated twice. In a 250 mL round bottom flask 1,1′-carbonyldiimidazole, CDI, (7.14 g) was dissolved with 100 mL DCM. A Teflon stir bar was inserted into the CDI solution and placed on a stir plate under nitrogen. The TrEG was dissolved with 50 mL DCM and slowly added to the stirred CDI solution, and the mixture was then stirred at room temperature for two hours under nitrogen. The reaction solution was transferred into a 250 mL separatory funnel and washed twice with 1 mM HCl followed by two brine solution washes. The organic solution was collected and dried with magnesium sulfate. The dried solution was filtered through a Whatman paper filter into a clean 250 mL round bottom flask and the DCM was rotary evaporated with mild heat (30° C.). A clear oil was collected (5.93 g).

### EXAMPLE 10

### Trimethylolpropane ethoxylate (20 EO)-tri(imidazolyl carbamate)

Trimethylolpropane ethoxylate (20/3 EO/OH), MW 1014, (10.14 g) was transferred to a 150 mL round bottom flask and dissolved with dichloromethane, DCM, (50 mL). The solvent was stripped off using a rotary evaporator and high temperature water bath. The stripping step was repeated twice. In a 1000 mL round bottom flask 1,1′-carbonyldiimidazole, CDI, (6.49 g) was dissolved with 250 mL DCM. A Teflon stir bar was inserted into the CDI solution and placed on a stir plate under nitrogen. The trimethylolpropane ethoxylate was dissolved with 100 mL DCM and slowly added to the stirred CDI solution, and the mixture was stirred at room temperature for two hours under nitrogen. The reaction solution was transferred into a 500 mL separatory funnel and washed twice with 1 mM HCl followed by two brine solution washes. The organic solution was collected and dried with magnesium sulfate. The dried solution was filtered through a Whatman paper filter into a clean 500 mL round bottom flask and the DCM was roto evaporated with mild heat (30° C.). A clear oil was collected (12.07 g).

### EXAMPLE 11

### Pentaerythritol ethoxylate (15 EO)-tetra(imidazolyl carbamate)

Pentaerythritol ethoxylate (15/4 EO/OH), MW 797, (11.96 g) was transferred to a 500 mL round bottom flask and dissolved with dichloromethane, DCM, (100 mL). The solvent was stripped off using a rotary evaporator and high temperature water bath. The stripping step was repeated twice. In a 1000 mL round bottom flask 1,1′-carbonyldiimidazole, CDI, (16.22 g) was dissolved with 200 mL DCM. A Teflon stir bar was inserted into the CDI solution and placed on a stir plate under nitrogen. The pentaerythritol ethoxylate was dissolved with 100 mL DCM and slowly added to the stirred CDI solution, and the mixture was stirred at room temperature for two hours under nitrogen. The reaction solution was transferred into a 500 mL separatory funnel and washed twice with 1 mM HCl followed by two brine solution washes. The organic solution was collected and dried with magnesium sulfate. The dried solution was filtered through a Whatman paper filter into a clean 500 mL round bottom flask and the DCM was roto evaporated with mild heat (30° C.). A clear oil was collected (15.89 g).

### EXAMPLE 12

### Preparation of a Shape Memory MD-Methacrylate Coil

A straight length of silicone tubing about 24 cm in length (0.029″ inner diameter (ID), 0.071″ outer diameter (OD), Helix Medical, Carpinteria, Calif.) was filled with a solution of MD-methacrylate at a concentration of 800 mg/mL (as prepared in Example 1) and photoinitiator 4,5-bis(4-benzoylphenyl-methyleneoxy)benzene-1,3-disulfonic acid, prepared as described in U.S. Pat. No. 6,278,018 (Example 1) at a concentration of 10 mg/mL, in deionized water using a syringe to inject the solution. The ends of the tube were pinched shut with clips and the tube with solution was wrapped tightly, but without stretching, around a larger 2.5 mm OD silicone tube with a steel rod inserted in the ID to keep the silicone tube straight. The coiled tube with solution was then fixture to the rod at both ends to keep it from uncoiling. The coiled tube with solution was then placed in a UV-light chamber The tubing was placed into an illumination system ((Dymax 400 watt power supplies with a 365-nm bulb; Dymax Corp.) to cure for 3 minutes with rotation. Once cured the tube was taken out and unwrapped. The silicone tube unwrapped mostly straight. The cured polymer matrix that was formed within the silicone tube was forced out by injecting a stream of water into one end using a syringe. The cured polymer matrix exited the other end in the shape of a coil about the size and shape of the wrapped tubing.

### EXAMPLE 13

### Preparation of a Silicone Mold of a Coil

A 0.014″ Teflon coated stainless steel wire was wrapped tightly around a 0.035″ steel mandrel to form a coil. The coil was stretched enough to produce an even gap in the coils. The coil was then inserted into a silicone tube larger than the OD of the coil and the tube was filled with curable RTV-silicone. The tube was filled with the RTV-silicone at a slow rate so that of all bubbles were removed centering the coil in the tube. This process embedded the Teflon-coated steel coil in RTV-silicone. The silicone was allowed to cure at room temperature and condition for 3 days. After curing, the Teflon coil was unscrewed from the cured silicone leaving a hollow void in the shape of a coil. Both ends of the mold were trimmed (cut) to ensure both ends of the coil void were open.

### EXAMPLE 14

### Preparation of a Shape Memory MD-Methacrylate Coil from a Mold

The MD acrylate/photoinitiator composition as prepared in Example 12 was used to fill the mold as prepared in Example 13. The mold with solution was placed in the UV chamber described in Example 12 and illuminated for 2 minutes with rotation to cure the solution and form a polymer matrix. The mold was taken out of the UV chamber and the formed polymer coil was forced out of the mold by a stream of water. The polymer coil came out of the mold as a coil of the same size and shape of the coil mold. The formed coil was rinsed in a bath of deionized water.

### EXAMPLE 15

### Reconfiguration and Dehydration of a Shape Memory MD-Methacrylate Coil

The coil prepared in Example 14 in hydrated state was placed on a flat Teflon surface. The coil was dried down to about 50-75% from its hydrated state and then a pair of Teflon rods were used to roll out the coil (both rods starting in-between the same coil gap and then moved in the opposite direction). Once the coil had been straightened, the rods were kept in contact with the coil to keep the coil in the straightened configuration until the coil was dry enough to remain straight without external pressure.

### EXAMPLE 16

### Intraocular Delivery, Rehydration, and Reconfiguration of a Shape Memory MD-Methacrylate Straightened Coil

The straightened coil formed in Example 15 was inserted into a 19 gauge needle. The needle was inserted into the harvested eyeball of a pig and the dry straightened coil was pushed through the needle into the vitreous using a metal mandrel as a plunger. The needle was withdrawn from the eye, and the dry straightened coil was allowed to hydrate in the vitreous. Within 2.5 minutes the coil had rehydrated and reverted back to its original coil shape.

### EXAMPLE 17

### Intraocular Delivery and Reconfiguration of a Rehydrated Shape Memory MD-Methacrylate Straightened Coil

A straightened coil as described in was Example 15 was inserted in dehydrated state and straightened configuration into a 19 gauge needle and then rehydrated with deionized water while in the needle. The needle was inserted into the harvested eyeball of a pig and a metal plunger was used to push out the hydrated coil into the vitreous of the eyeball. The coil held in the straightened configuration by the needle came out the end of the needle and immediately took the shape of a coil (i.e., the coil emerged from the end of the needle like a cork-screw). The polymer coil was suspended in the vitreous and did not move or sink from its delivered position.

### EXAMPLE 18

### Preparation of Biodegradable Shape Memory Coils from a Mold

The MD acrylate/photoinitiator composition as prepared in Example 12 was prepared, with the addition of Bovine Serum Albumin (BSA) at a concentration in the range of about 30-40%.

The subsequent solution was injected into a silicone mold of a coil and UV cured. The cured polymer/protein matrix was expelled from the mold and retained the shape of the coil. The coil was rinsed in deionized water, then taken out and placed in a petri-dish to air dry.

### EXAMPLE 19

### Preparation of Biostable Shape Memory Coil

A solution of PEG-triacrylate macromer (as described in United States Patent Publication No. US-2004-0202774-A1) and photoinitiator as described in Example 12 at 33% and 6.6 mg/mL, respectively, in DI-water was injected into a 35-37 cm length of silicone tubing (0.029″ ID) from Helix Medical and then the ends were sealed with alligator clips. The tube was then wrapped compactly around a 2.5 mm diameter silicone tube (with a steel mandrel inserted for support). The ends of the tube with solution were attached to the silicone/steel tube to keep coiled. The system was then placed in a UV-light chamber and illuminated for 2-minutes (with rotation) to cure the polymer matrix. The tube with cured solution/matrix was allowed to unwrap and was mostly straight with some minor waviness. Water was forcefully injected into one end of the tubing to force out the UV-cured polymer matrix, which exited the other end of the tubing as a coil similar in shape and size as the wrapped silicone tubing.

### EXAMPLE 20

### Preparation of Biostable Shape Memory Tapered Coil

A solution of PEG-triacrylate macromer and photoinitiator as described in Example 19 was injected into a 35-37 cm length of silicone tubing (0.029″ ID) from Helix Medical and then the ends were sealed with alligator clips. The tube was then wrapped compactly around the distal end of a plastic pipette (straight to tapered larger). The ends of the tube with solution were attached to the plastic pipette section to keep coiled. The system was then placed in a UV-light chamber and illuminated for 2-minutes (with rotation) to cure the polymer matrix. The tube with cured solution/matrix was allowed to unwrap and was mostly straight with some minor waviness. Water was forcefully injected into one end of the tubing to force out the UV-cured polymer matrix, which exited the other end of the tubing as a coil similar in shape and size as the wrapped silicone tubing (tapered coil).

### EXAMPLE 21

### Preparation of Biostable Shape Memory Article in “Figure-8” Configuration

A solution of PEG-triacrylate macromer and photoinitiator as described in Example 19 was injected into a 35-37 cm length of silicone tubing (0.029″ ID) from Helix Medical and then the ends were sealed with alligator clips. The tube was then wrapped or weaved relatively loosely around a 0.035″ steel mandrel bent in the shape of a “U” with a gap of approximately 1.3 cm (forming a series of figure 8's). The ends of the steel “U” were inserted into a silicone plug to keep from collapsing. The ends of the tube with solution were attached to the steel mandrel to keep wrapped. The system was then placed in a UV-light chamber and illuminated for 2-minutes (with rotation) to cure the polymer matrix. The tube with cured solution/matrix was allowed to unwrap and was mostly straight with some minor waviness. Water was forcefully injected into one end of the tubing to force out the UV-cured polymer matrix which exited the other end of the tubing as a series of figure 8's that would basically lay down on one another.

### EXAMPLE 22

### Preparation of Biostable Shape Memory Article in Knotted Configuration

A solution of PEG-triacrylate macromer and photoinitiator as described in Example 19 was injected into a 35-37 cm length of silicone tubing (0.029″ ID) from Helix Medical and then the ends were sealed with alligator clips. The tube was then tied into a series of eight loose knots, each with a rough diameter of 1-cm. The knotted tubing was allowed to hang vertically (length of about 10-cm) while being illuminated in a UV-light chamber for 2-minutes (with rotation) to cure the polymer matrix. Without untying the tube, water was forcefully injected into one end of the tubing to force out the UV-cured polymer matrix which exited the other end of the tubing and immediately assumed a configuration similar in shape and size as the knotted silicone tubing, but without the knots actually being looped as true knots (all of the bends were exactly where they were in the knotted silicone).

### EXAMPLE 23

### Preparation of MD-Methacrylate Cylinder

A section of a 1 mm diameter Teflon rod was dip coated using a solution of MD-methacrylate and photoinitiator as describe in as prepared in Example 12. The rod was dipped into the solution at 0.5 cm/sec a distance of 3.1 cm and allowed to dwell for 15 sec before being pulled out at 0.1 cm/sec. The coated rod was immediately placed in a UV-illumination chamber to cure (with rotation) for 45 sec. The process was repeated 2 more times for a total of 3 coats of the matrix. The cured rod/coating was then soaked in DI-water for 10-minutes. After soaking the cured hydrated polymer matrix was pulled off of the Teflon rod by applying moderate finger pressure to break any mechanical adhesion to the rod. The polymer matrix slipped off the rod as a tube. Upon air drying the polymer tube shrank uniformly about 14%.

### EXAMPLE 24

### Reconfiguration and Dehydration of MD-Methacrylate Cylinder

The hydrated cylinder from Example 23 was placed in a silicone half pipe prepared by longitudinally bisecting a length of 2 mm diameter silicone tubing. Pressure was applied longitudinally across the hydrated cylinder with a 0.2 mm diameter wire. The cylinder slowly collapsed inward without fracturing. The wire was removed and the collapsed cylinder was allowed to dehydrate completely. The collapsed cylinder had a diameter approximately half that of the uncollapsed cylinder. When the cylinder was rehydrated it slowly (approximately 2.0 minutes) assumed the form of the original uncollapsed cylinder.

### EXAMPLE 25

### Mold for Shape Memory Cylinder Preparation

A mold (“tube with in a tube” design) for fabrication of a shape memory cylinder was prepared. A smaller diameter silicone tube (Dow Corning Q7-4750; ID=3.35 mm; OD=4.65 mm; Helix Medical (Carpinteria, Calif.), Cat.# 60-011-11;) was positioned inside a larger diameter silicone tube (Dow Corning Q7-4750; ID=6.35 mm; OD=11.11 mm Cat.# 60-011-25) using a section cut from the ribbed top portion of a 200 μl pipette tip as a spacer (the small tube fit snuggly inside the pipette tip section and the outer tube fit snuggly over the pipette tip section) between the tubes on both ends to create a uniform gap or void circumferentially between the tubes. One end (the top end) was allowed to have perforations (caused by the ribbing of the pipette tip section) between the spacer and the tubing to allow air to escape. The opposite end (the bottom) was sealed by wrapping Parafilm™ over and around the end of the tubing and the section of pipette tip. One small puncture was created in the outer silicone tubing wall just above the bottom end's spacer/ring.

### EXAMPLE 26

### Shape Memory Cylinder Preparation

Using the mold fabricated according to Example 25, a biodegradable shape memory cylinder was prepared from reagents forming an inter penetrating network (IPN) of biodegradable polymeric material. The IPN was formed by combining mutually reactive polymeric components (PEG-diimidazolyl carbonate and aminated polyalditol), and a UV-induced free radical reactive polymeric component (Maltodextrin-methacrylate).

A first solution (Solution A) was prepared that included free-radical polymerizable biodegradable macromer and monomeric components, and a photoinitiator. Solution A was prepared by adding the following to an 8-ml clear glass vial 650 mg of MD-methacrylate (see example 1) and 325 mg of diacetone acrylamide (DAAM) (Lot #B5176B; Alfa Aesar, Ward Hill, Mass.) and 1.3 ml of a 5 mg/ml 4,5-bis(4-benzoylphenylmethyleneoxy)benzene-1,3-disulfonic acid solution, prepared as described in U.S. Pat. No. 6,278,018 (Example 1) solution in deionized water. This solution was vortexed for 5 minutes and then sonicated for 20 seconds to remove air bubbles.

A second solution (Solution B) was prepared by adding to a second vial, 556 mg of PEG₁₀₀₀-diIC (see example 6) along with 560 μl of the Solution A. This solution was vortexed for 10 minutes and then sonicated for 60 seconds to remove air bubbles.

A third solution (Solution C) of PD60—NH₂ (see example 2) at approximately 950 mg/ml (pH adjusted to 7) was prepared.

To the vial containing the Solution B was added approximately 950 μl of the viscous Solution C and the mixture was vortexed for 2 minutes, then sonicated for 2 minutes to remove the majority of the bubbles. The solution was then drawn up into a 1 ml syringe. A 200 μl pipette tip was then cut near the top to fit tightly onto the end of the syringe. The curable solution was then injected into the mold as prepared in Example 25 via a syringe by inserting the very tip of a 200 μl pipette tip through the puncture near the bottom of the mold. The mold was positioned so that as the solution entered the mold the air would escape through the top end allowing the solution to fill the mold bubble free.

The solution filled mold was placed upright in a sealed container and allowed to cure overnight at room temperature. The next day the mold was taken out of the sealed container and placed in a UV light box (1 Dymax lamp—400 watt power supply—facing down towards the sample which lays on a flat surface) and illuminated for a total of 2 minutes. The mold was rotated ¼ turn every 30 seconds. and the solution was injected into the silicone tubing mold

The spacers were removed from both ends of the mold and the smaller inner silicone tube was pulled out of the mold without damage to the formed matrix. The mold was then left open at room temperature and the matrix was allowed to air dry and shrink, thus pulling away from the silicone wall as it shrank and was easily pushed out once dry. The cured, dry matrix tube was relatively uniform and pliable. It shrank uniformly approximately 20% in size.

### EXAMPLE 27

### Shape Memory Coil with Therapeutic Microparticles

A biodegradable shape memory coil containing anti-proliferative loaded microparticles was prepared.

The microparticles were prepared using poly(L-lactide-co-caprolactone-co-glycolide; PLLCG) Lot 08723kA (Sigina) and rapamycin lot ASW-106 (Sirolimus, Wyeth). pLLCG solutions were made at 500 mg in 10 ml of dichloromethane. 25 mg of Rapamycin was added to 2 ml of the pLLCG solution to form a 25% w/w polymer solution. The polymer solution was thoroughly mixed into 10 ml of aqueous polyvinylalcohol (PVA) solution 0.2% w/w. The mixture was put on ice and further dispersed by ultra sonication (probe) using pulsated sonication: 2 secs pulse and 0.5 sec pause. The dispersion was poured into 100 ml 0.2% PVA solution, handmixed for 1.5 minutes and stirred at room temperature for 3 hours. The microspheres were isolated by centrifugation (10 k RPM for 5 minutes). The samples were then frozen and lyophilized. The yield was 58.4 mg (47%).

For the body member, a solution was prepared by adding 1000 mg of PD60-A (polyalditol acrylate was prepared as described in Example 18 of U.S. Patent Publication 2007/0065484 (Chudzik et al.; App Ser. No. 11/525,006; pub. Date Mar. 22, 2007) to an 8-ml clear glass vial followed by 2.5 ml of a 5 mg/ml 4,5-bis(4-benzoylphenylmethyleneoxy)benzene-1,3-disulfonic acid solution in deionized water which was added in 0.5-1.0 ml increments, with vortexing of the solution/vial in between additions. This solution was then allowed to mix on an orbital shaker for 4 hours, at which time 500 mg of DAA was added to the mixture and the solution was vortexed again to break up the solids. The solution was then allowed to mix on an orbital shaker overnight. The final volume of the prepared solution was estimated to be 4.5 ml±0.1 ml.

To this solution/vial was then added 33.2 mg of the pLLCG particles with a targeted 25% drug load. This solution was vortexed and sonicated intermittently for 40 minutes to disperse the microparticles and remove the bubbles.

The solution with suspended microparticles was then injected via a syringe system into a 44 cm length of silicone tubing with an inner diameter of 1.2 mm and an outer diameter of 2.45 mm. The ends of the filled tubing were then plugged and the tubing was wrapped/coiled tightly around a cylindrical shaft with an outer diameter of 7 mm and fixtured in place at both ends. The solution filled coil was then UV illuminated between two opposing Dymax lamps (400 watt power supplies) for 2 minutes with rotation.

Once UV cured, a layer of Parafilm was wrapped around the mid-section of the coil, leaving the ends exposed. The plugs in each end were removed and a stream of water was forced into one end of the silicone tube via a syringe system, which forced the cured matrix to exit the opposite end of the silicone tube. The matrix took the shape of the formed coil upon exiting the tubing.

The formed coil matrix is straightened by hanging the coil from one end and attaching a small weight to the other end, thereby stretching the coil to a linear configuration. The re-configured coil is allowed to dry in the linear configuration. Upon drying the re-configured coil remains straight without force being applied to the coil to keep it in the linear configuration. Upon re-hydratation the re-configured coil reverts back to it original coil shaped configuration.

What is claimed is:

1. An insertable medical article comprising a body member comprising a crosslinked matrix of hydrophilic polymer having a molecular weight of 100,000 Da or less, wherein the body member is capable of undergoing a shape memory transition from a second configuration to a first configuration upon insertion of the article at a target site in a subject.

2. The insertable medical article of claim 1 wherein the hydrophilic polymer has a molecular weight of 50,000 Da or less.

3. The insertable medical article of claim 2 wherein the hydrophilic polymer has a molecular weight of 25,000 Da or less.

4. The insertable medical article of claim 3 wherein the hydrophilic polymer has a molecular weight in the range of 1000-10,000 Da.

5. The insertable medical article of claim 1 wherein the matrix comprises hydrophilic polymer that is crosslinked via polymerized groups.

6. The insertable medical article of claim 1 wherein the matrix comprises a hydrophilic polymer and a hydrophilic component crosslinked via first and second reacted groups, wherein the first and second reacted groups are pendent from the hydrophilic polymer and a hydrophilic component, respectively.

7. The insertable medical article of claim 6 wherein the first reactive group is present on the polysaccharide in an amount to provide a degree of substitution of 1.0 or less.

8. The insertable medical article of claim 7 wherein the first reactive group is present on the polysaccharide in an amount to provide a degree of substitution in the range of 0.2 to 0.3.

9. The insertable medical article of claim 6 wherein the first or second reacted group is an amine group.

10. The insertable medical article of claim 6 wherein the second component is non-biodegradable.

11. The insertable medical article of claim 6 wherein the second component has a MW of 10,000 Da or less.

12. The insertable medical article of claim 6 wherein the second component has a MW of 3,500 Da or less.

13. The insertable medical article of claim 6 wherein the hydrophilic component comprises an alkoxyalkane.

14. The insertable medical article of claim 8 wherein the alkoxyalkane is selected from the group consisting of tri(ethylene glycol), tetra(ethylene glycol), poly(ethylene oxide) (PEO), poly(ethylene glycol), and poly(propylene oxide) (PPO).

15. The insertable medical article of claim 6 wherein the hydrophilic component is a liquid at 25° C.

16. The insertable medical article of claim 1 wherein the hydrophilic polymer is a natural biodegradable polymer.

17. The insertable medical article of claim 12 wherein the natural biodegradable polymer is selected from the group consisting of maltodextrin, amylose, polyalditol, and cyclodextrin.

18. The insertable medical article of claim 1 wherein the body member comprises 15% or greater by weight hydrophilic polymer.

19. The insertable medical article of claim 1 wherein the second configuration is linear.

20. The insertable medical article of claim 1 wherein the first configuration comprises a coil configuration.

21. The insertable medical article of claim 1 where the body member is in the form of a cylinder.

22. The insertable medical article of claim 1 comprising a body member for use in the vasculature.

23. The insertable medical article of claim 1 wherein the body member in the first configuration can exert pressure on tissue at the target site.

24. The insertable medical article of claim 23 comprising a stent.

25. The insertable medical article of claim 1 comprising a body member for use within the eye.

26. The insertable medical article of claim 1 comprising a bioactive agent.

27. The insertable medical article of claim 26 comprising a bioactive agent selected from the group consisting of polypeptides, polynucleotides, and polysaccharides.

28. The insertable medical article of claim 26 comprises a bioactive agent having a molecular weight of 10,000 or greater.

29. The insertable medical article of claim 1 wherein the body member is the second configuration has a second hydration state, and is capable of undergoing a shape memory transition to a first configuration upon insertion of the article at a target site in a subject which increases the hydration of the body member to a first hydration state.

30. The insertable medical article of claim 29 wherein in the second hydration state, the body member comprises an amount of water of 35% or less.

31. A method for preparing an insertable medical article comprising a body member having a shape memory property, the method comprising steps of:
providing a composition in a first configuration, the composition comprising:
a hydrophilic polymer having a molecular weight of 100,000 Da or less, and
a reactive group; and
causing reaction of the reactive group thereby forming a matrix of hydrophilic polymer and fabricating the body member in the first configuration,
wherein the body member is capable of reverting from a second configuration to the first configuration upon or following insertion at a target site in a subject.

32. The method of claim 31 where, in the step of providing, the composition comprises a hydrophilic polymer at a concentration of 250 mg/mL or greater.

33. The method of claim 31 where, in the step of providing, the composition comprises 50 wt % water or less.

34. The method of claim 33 comprising a step of removing at least a portion of water from the body member to stabilize the body member in the second configuration.

35. The method of claim 34 where, in the step of removing, at least 50% of the liquid present in the body member is removed.

36. The method of claim 34, wherein the step of providing a composition in a first configuration comprises a substep of mixing the hydrophilic polymer having a molecular weight of 100,000 Da or less comprising a first reactive group, and a second component that is hydrophilic and that comprises a second reactive group, wherein the first and second groups are specifically reactive and provide a crosslinked hydrophilic polymeric matrix upon mixing, thereby fabricating the body member in the first configuration.

37. The method of claim 36 wherein the hydrophilic polymer and second component react to form a first polymeric network and the composition further comprises a second hydrophilic polymer comprising a pendent polymerizable group, and the method further comprises a step of treating to cause polymerization of the second hydrophilic polymer which forms a second polymeric network that penetrates the first polymeric network, which forms the body member.

38. A method for treating a target site within a subject, comprising the steps of
obtaining an insertable medical article comprising a body member comprising a crosslinked matrix of hydrophilic polymer, wherein the body member is capable of undergoing a shape memory transition from a second configuration to a first configuration;
delivering the article to a target site in the body in the second configuration;
allowing the body member to revert from the second configuration to the first configuration at the target site,
wherein the body member in the first configuration exerts force on a tissue at the target site.

39. The method of claim 38, wherein the step of allowing the body member becomes hydrated to promote change to the first configuration.

40. A method for delivering a bioactive agent to a subject, comprising the steps of
obtaining an insertable medical article comprising a body member comprising a crosslinked matrix of hydrophilic polymer having a molecular weight of 100,000 Da or less and a bioactive agent, wherein the body member is capable of undergoing a shape memory transition from a second configuration to a first configuration;
delivering the article to a target site in the body in the second configuration;
allowing the body member to revert from the second configuration to the first configuration at the target site; and
allowing release of the bioactive agent from the body member at the target site.

41. An insertable medical article comprising a body member formed of a hydrophilic matrix comprising a first polymeric network that penetrates a second polymeric network, wherein the body member is capable of undergoing a shape memory transition to the first configuration from a second configuration.

42. A system for delivering to a portion of a body an insertable medical article comprising a body member having a shape memory property comprising (a) an insertable medical article comprising a body member comprising a crosslinked matrix of hydrophilic polymer, wherein the body member is capable of undergoing a shape memory transition from a second configuration to a first configuration upon insertion of the article at a target site in a subject and (b) an insertion instrument capable of holding the article in the second configuration for delivery of the article to a portion of the body.
